(19) United States
(12) Patent Application Publication
(10) Pub. No.: US 2004/0110843 A1
(43) Pub. Date: Jun. 10, 2004

(54) Methods of treatment of male erectile dysfunction

(75) Inventors: James L. Yeager, Lake Forest, IL (US); Nadir Buyuktimkin, Robbinsville, NJ (US); Servet Buyuktimkin, Robbinsville, NJ (US)

Correspondence Address: Roger P. Zimmerman, BOWDITCH & DEWEY, LLP, 161 Worcester Road, P.O. Box 9320, Framingham, MA, 01701-9320, US

(73) Assignee: NexMed (Holdings), Inc.

(21) Appl. No.: 10/656,906

(22) Filed: Sep. 5, 2003

Related U.S. Application Data
(63) Continuation-in-part of application No. 10/236,485, filed on Sep. 6, 2002.
Application No. 10/236,485 is a continuation-in-part of application No. 09/947,617, filed on Sep. 6, 2001, now Pat. No. 6,693,135.
Application No. 09/947,617 is a continuation-in-part of application No. 09/480,738, filed on Jan. 10, 2000, now Pat. No. 6,323,241.
Application No. 09/947,617 is a continuation-in-part of application No. PCT/US01/00852, filed on Jan. 10, 2001.
(60) Provisional application No. 60/483,278, filed on Jun. 27, 2003.

(51) Int. Cl. A61K 31/557

(52) U.S. Cl. 514/573

(57) ABSTRACT

The invention provides methods for the treatment of erectile dysfunction in a patient suffering from a co-morbid condition comprising placing in the fossa navicularis of the patient an erection-inducing amount of a semi-solid vasoactive prostaglandin composition.

### RELATED APPLICATIONS

This application claims the benefit of U.S. Provisional Application No. 60/483,278, filed Jun. 27, 2003, and is a continuation-in-part of co-pending application Ser. No. 10/236,485, filed Sep. 6, 2002, which is a continuation-in-part of co-pending application Ser. No. 09/947,617, filed Sep. 6, 2001, which is a continuation-in-part of application Ser. No. 09/480,738, now U.S. Pat. No. 6,323,241, and a continuation-in-part of International Application Serial No. PCT/US00/00852, filed Jan. 10, 2001, the entire contents of which are incorporated herein by reference.

### TECHNICAL FIELD OF THE INVENTION

This invention relates to the methods for treatment of erectile dysfunction in patients suffering from a co-morbid condition.

### BACKGROUND OF THE INVENTION

The term “impotence” has been used to signify the inability of the male to attain and maintain erection of the penis sufficient to permit satisfactory sexual intercourse. The term “erectile dysfunction” has been suggested as a more precise term “to signify an inability of the male to achieve an erect penis as part of the overall multifaceted process of male sexual function.” Droller, M. J. et al. Impotence. Consensus Development Conference Statement, National Institutes of Health (1993).

Erectile dysfunction may result from psychological causes (psychogenic erectile dysfunction) or organic causes or a combination of both. Organic causes include physiological, nervous, vascular and hormonal pathologies or a combination thereof.

The normal physiology of an erection involves nerve impulses that signal certain muscles to relax. These muscles, when contracted, restrict blood flow through arteries in the penis. When relaxed, the muscles permit a significant increase in blood flow. The increased blood flow engorges three groups of erectile tissue within the penis with blood and the penis becomes less flaccid. The engorged erectile tissue and the muscle structure of the penis depress adjacent veins, restricting the flow of blood out of the penis. The restriction of blood flow out of the penis increases and sustains the erection.

Deficiencies of some hormones, such as testosterone, or elevation of others, such as prolactin, can cause erectile dysfunction. Many drugs, such diuretics, antihypertensives, anticonvulsants, narcotics, alcohol, and psychotropic drugs may cause erectile dysfunction as a side effect. Murray, F. T. et al. Amer. J. Medical Sci. 309: 99-109 (1995).

Damage to nerves and blood vessels may also provide an organic cause for erectile dysfunction. Disease processes may involve several aspects. For example, diabetes, which causes damage to both nerves and blood vessels, can cause erectile dysfunction. A significant percent of all diabetic men will suffer from erectile dysfunction. While diabetes mellitus is a common risk factor in erectile dysfunction (ED), the pathogenesis of ED in diabetes is not completely understood (Sullivan, M. E., et al., Alterations in endothelin B receptor sites in cavernosal tissue of diabetic rabbits: potential relevance to the pathogenesis of erectile dysfunction. J Urol. 1997 158(5):1966-72). ED in diabetes may be one aspect of vascular disease associated with diabetes (Sairam, K., et al., Prevalence of undiagnosed diabetes mellitus in male erectile dysfunction. BJU Int. 2001 88(1):68-71; Sullivan, M. E., et al. Nitric oxide and penile erection: is erectile dysfunction another manifestation of vascular disease? Cardiovasc Res. 1999 Aug. 15;43(3):658-65) Microvasculopathy is one of the characteristics of diabetes. Studies have suggested a link between diabetes, erectile dysfunction and endothelial cell dysfunction (De Angelis, L., et al., Erectile and endothelial dysfunction in Type II diabetes: a possible link. Diabetologia. 2001 44(9): 1155-60; Burchardt, T., et al., Reduction of endothelial and smooth muscle density in the corpora cavernosa of the streptozotocin induced diabetic rat. J Urol. 2000 164(5):1807-11; Hopfner, R. L., & Gopalakrishnan, V., Endothelin: emerging role in diabetic vascular complications. Diabetologia. 1999 42(12):1383-94). In other studies, erectile dysfunction has been found to be predominant among patients affected by cardiovascular disease or diabetes mellitus, and the presence of cardiovascular disease increased the risk of erectile dysfunction (Sasayama, S., et al. Men's Health Study. Epidemiology of Erectile Dysfunction and Cardiovascular Disease, Circ. J., 2000; 67:656-659).

Radical retropubic prostatectomy (RPP) has been the standard treatment for organ/specimen-confined prostate cancer for several decades, yet erectile dysfunction in selected series is still reported as high as 90% after this procedure, with surgical technique and experience dominant variables influencing outcome (Zippe, C. D., et al., Management of erectile dysfunction following radical prostatectomy, Current Urology Reports, 2: 495-503 (2001). Age is also a factor. Although about 50-70% of younger men regain potency after nerve sparing radical prostatectomy, the potency recovery rate in patients over 70 years of age is less than 10% (Catalona, W. J., et al., Nerve-sparing radical prostatectomy: evaluation of results after 250 patients. J. Urol. 1990; 143:538-43; discussion 44; Quinlan, D. M., et al., Sexual function following radical prostatectomy: influence of preservation of neurovascular bundles. J. Urol. 1991; 145:998-1002).

Thus, most men need treatments for erectile dysfunction to be sexually active following radical prostatectomy. Treatments using vacuum constriction devices, intracorporeal injections of vasoactive drugs, and transurethral vasodilators, have reported response rates of 50% to 70%, but poor long-term compliance, having discontinuation rates of nearly 50% at one year.

Methods proposed for the treatment of erectile dysfunction have included external devices, sex therapy, surgical implantation of internal prostheses, injection of drugs directly into the penis and topically applied medications. None of these approaches is entirely effective.

External devices include tourniquets (see U.S. Pat. No. 2,818,855) and externally applied vacuum erection aids. While some clinicians consider externally applied erection aids as a first option for treatment, some patients are unwilling to use such devices. O'Keefe, M., et al. Medical Clinics of North America 79: 415-434 (1995).

Symptomatic sex therapy was originally found to be effective by Masters and Johnson, but later studies have not shown as impressive results. Freudian therapy does not appear to patients to be an attractive alternative. Vickers, M. A., et al. J. Urology 149: 1258-1261 (1993).

Surgically implanted mechanical devices, such as hinged or solid rods and inflatable, spring driven or hydraulic prostheses have been used for some time. Several penile prostheses have been described that are pliable plastic elements with constant rigidity. However, these prostheses are continuously rigid and can cause discomfort for the patient. Other types of penile prostheses include surgically positioned pumps for creating high pressure of liquid in the elastic silicon mantle. Implantation of these complex devices requires implanting several components into the patient's body, for example, the reservoir with liquid, pump, several valves, connecting pipes, and the like, in addition to those components implanted directly into the penis. Other penile prostheses use an external source of electricity and a source of an alternating magnetic field changing with the frequency of 50 to 1000 Hz that influences the internal element located in the penis. This element senses the magnetic field and causes liquid in the inner reservoir to move from the reservoir into the elastic mantles located in the corpora cavernosa, and causes the penis to erect. Some disclosures describe prostheses including a permanent magnet that makes seesaw movements under the influence of this field, which in turn causes liquid from the reservoir to pump into the elastic mantles, thus serving as an internal element sensing alternating magnetic field of the external source. However, while such prostheses can provide adequate rigidity for intercourse, patients and the patients' partners have been reported to indicate unmet expectations with their penile prostheses. Case reports have recounted the results of treating a single patient with intracavernosal injections of PGE₁ (Keogh, E. J. and Earle, C. M., Int. J. Impotence Res., 4:113, 1992) or with MUSES (Chew, K. K., & Stuckey, B. G. A., Int. J. Impotence Res., 12: 195-196, 2000) in an attempt to alleviate dissatisfaction with a penile prosthesis.

The administration of erection effecting and enhancing drugs is taught in U.S. Pat. No. 4,127,118 to LaTorre. This patent teaches a method of treating male impotence by injecting into the penis an appropriate vasodilator, in particular, an adrenergic blocking agent or a smooth muscle relaxant to effect and enhance an erection.

More recently, U.S. Pat. No. 4,801,587 to Voss et al. teaches the application of an ointment to relieve impotence. The ointment consists of the vasodilators papaverine, hydralazine, sodium nitroprusside, phenoxybenzamine, or phentolamine and a carrier to assist absorption of the primary agent through the skin. U.S. Pat. No. 5,256,652 to El-Rashidy teaches the use of an aqueous topical composition of a vasodilator such as papaverine together with hydroxypropyl-β-cyclodextrin.

The biochemical, physiological, and clinical effects of cyclic guanosine 3′,5′-monophosphate specific phosphodiesterase (cGMP-specific PDE) inhibitors suggest their utility in a variety of disease states in which modulation of smooth muscle, renal, hemostatic, inflammatory, and/or endocrine function is desired. One isozyme in particular, the type 5 cGMP-specific phosphodiesterase (PDE5) is the major cGMP hydrolyzing enzyme in vascular smooth muscle, and its expression in penile corpus cavernosum has been reported. Thus, PDE5 is an attractive target in the treatment of sexual dysfunction. A selective inhibitor of PDE5, sildenafil, has been available and marketed in a pharmaceutical product VIAGRA®. While sildenafil has obtained significant commercial success, it has fallen short due to its significant adverse side effects, including facial flushing (10% incidence rate). Adverse side effects limit the use of sildenafil in patients suffering from visual abnormalities, hypertension, and, most significantly, by individuals who use organic nitrates (Welds et al., Amer. J. of Cardiology, 83 (5A), pp. 21(C)-28(C) (1999)). The use of sildenafil in patients taking organic nitrates is believed to cause a clinically significant drop in blood pressure which could place the patient in danger. Accordingly, the package label for sildenafil provides strict contraindications against its use in combination with organic nitrates (e.g., nitroglycerin, isosorbide mononitrate, isosorbide dinitrate, erythrityl tetranitrate) and other nitric oxide donors in any form, either regularly or intermittently, because sildenafil potentiates the hypotensive effects of nitrates. See C. R. Conti et al., Amer. J. of Cardiology, 83(5A), pp. 29C-34C (1999). Thus, even with the availability of sildenafil, there remains a need to identify improved pharmaceutical products that are useful in treating sexual dysfunction in patients with co-morbid conditions such as hypertension.

One approach has been to seek other oral phosphodiesterase-5 inhibitors with greater selectivity that would hopefully lack the undesirable side effects of sildenafil. See U.S. Pat. No. 6,451,807. Two such oral phosphodiesterase-5 inhibitors are vardenafil (Levitra™) and tadalafil (Cialis™). Early evidence indicates that these other two phosphodiesterase-5 inhibitors are similar to sildenafil in interactions with concomitant therapy in angina patients receiving nitrate treatment (contraindication) and hypertension patients receiving alpha-blockers. See Gresser, U., & Gleiter, C. H., Erectile dysfunction: Comparison of efficacy and side effects of the PDE-5 inhibitors sildenafil, vardenafil and tadalafil. Review of the literature, European J, Med. Res., 2002, 7: 435-446.

However, this approach of looking for more specific inhibitors does not necessarily solve another problem with oral phosphodiesterase-5 inhibitor treatment, the lack of response to treatment in a significant fraction of erectile dysfunction patients. Alternatively, another class of drugs acting on another therapeutic target can be sought for treatment of erectile dysfunction in patients who are unresponsive to oral phosphodiesterase-5 inhibitor therapy. It would also be desirable to have alterative treatments for patients suffering from additional co-morbid conditions, such as hypertension treated with alpha blockers, or angina treated with nitrates, for which for oral phosphodiesterase-5 inhibitor therapy is contraindicated or subject to warnings.

Prostaglandin E₁ is a derivative of prostanoic acid, a 20-carbon atom lipid acid, represented by the formula:

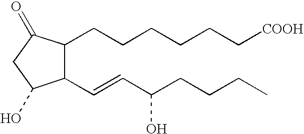

and is commercially available, e.g., from Chinoin Pharmaceutical and Chemical Works Ltd. (Budapest, Hungary) under the designation “Alprostadil USP,” and from Pharmacia & Upjohn under the designation “Caverject”. Prostaglandin E₁ complexed with alpha-cyclodextrin is available as alprostatil alfadex from Ono Pharmaceuticals (Japan) and in an injectable form under the designation “Edex®” or “Viradex®” from Schwarz Pharma (Germany).

In one commercially available dosage form (MUSE®, Vivus, Menlo Park Calif.), alprostadil is administered in a pellet deposited in the urethra using an applicator with a hollow stem 3.2 cm in length and 3.5 mm in diameter (Padma-Nathan, H., et al., N. Engl. J. Med., 336: 1-7 (1997), see especially FIG. 1). In the home treatment portion of the Padma-Nathan et al. study, 32.7% of the patients (10.8% of administrations) receiving MUSE® complained of penile pain and 5.1% experienced minor urethral trauma, compared to 3.3% and 1.0%, respectively, of the patients receiving placebo. Frequency of report of these side effects has varied in subsequent studies: MUSE®) producing penile pain in 17-23.6% of administrations, compared to 1.7% with placebo and minor urethral bleeding reported by 4.8% of patients (Peterson, C. A., et al., J. Urol., 159: 1523-1528 (1998)). In a study on a European population, 31% MUSE® patients reporting penile pain or burning sensations, 4.8% reporting urethral bleeding, and 2.9% reporting severe testicular pain (Porst, H., Int. J. Impot. Res., 9:187-192 (1997)). The percent of patients responding to MUSE® treatment, defined as having at least one erection considered sufficient for intercourse, has been reported to be 43% (Porst, 1997), 65.9% (Padma-Nathan et al., 1997) and 70.5% (Peterson et al., 1998), although published editorial comment has suggested that the percent of patients responding in the latter two studies is more properly reported as 30-40% (Benson, G., J. Urol., 159: 1527-1528 (1998).

Intraurethral application of a preparation of 1 mg prostaglandin E₁ in phosphatidylcholine liposomes in 1 ml polyoxyethylene glycol has been reported to be less effective than intracavernosal injection of prostaglandin E₁ (Englehardt, P. F., et al., British J. Urology, 81: 441-444, 1998). No ED patients receiving the liposomal preparation achieved complete penile rigidity, and only 6 of 25 patients achieved an erection adequate for vaginal penetration. In contrast, intracavernosal injection of prostaglandin E₁ produced erections adequate for vaginal penetration or complete rigidity in 23 of 25 of the same ED patients. The authors suggested that the transurethral effect of the prostaglandin E₁ probably arises by diffusion of prostaglandin E₁ first into the corpus spongiosum and then into the corpus cavernosum.

While the above mechanical and pharmaceutical treatments have focused on producing adequate penile rigidity, even when the treatments succeed in producing adequate rigidity, the satisfaction of the patient and the patient's sexual partner is often less than adequate. Patients discontinue medical treatments that produce rigidity, such as intracavenosal injections or transurethral suppositories because of painful side effects. Penile implants may produce rigidity, but insufficient tumescence. In particular, lack of tumescence of the glans penis is a recognized source of dissatisfaction for both the patient and the sexual partner (See, e.g., U.S. Pat. No. 6,418,934; Chew & Stuckey, 2000).

### SUMMARY OF THE INVENTION

The invention provides pharmaceutical methods for the treatment of erectile dysfunction in a patient suffering from at a co-morbid condition comprising placing in the fossa navicularis of a patient in need of such treatment an erection-inducing amount of a semi-solid composition comprising a vasoactive prostaglandin and a penetration enhancer. The co-morbid condition is at least one of diabetes mellitus, hypertension, cardiac disease, recovery from prostatectomy, or erectile dysfunction unresponsive to oral phosphodiesterase-5 inhibitor therapy.

In one preferred embodiment, the present invention provides methods for the treatment of erectile dysfunction in an individual suffering from the co-morbid condition diabetes mellitus. In another preferred embodiment, the present invention provides methods for the treatment of erectile dysfunction in an individual suffering from the co-morbid condition hypertension. In yet another preferred embodiment, the present invention provides methods for the treatment of erectile dysfunction in an individual suffering from the co-morbid condition cardiac disease. In still another preferred embodiment, the present invention provides methods for the treatment of erectile dysfunction in an individual recovering from prostatectomy. In a further preferred embodiment, the present invention provides methods for the treatment of an individual suffering from erectile dysfunction unresponsive to oral phosphodiesterase-5 inhibitor therapy.

A patient suffering from erectile dysfunction and at least one co-morbid condition such as diabetes mellitus, hypertension, cardiac disease, recovery from prostatectomy or erectile dysfunction unresponsive to oral phosphodiesterase-5 inhibitor therapy can be effectively treated by placing in the fossa navicularis of the patient an erection inducing amount of a semi-solid vasoactive prostaglandin composition, which contains a dose of about 0.05 mg to about 0.8 mg of a vasoactive prostaglandin, a penetration enhancer, a polymeric thickener selected from the group consisting of a polysaccharide gum and a polyacrylic acid polymer, a lipophilic component that is selected from the group consisting of an aliphatic C₁ to C₈ alcohol, an aliphatic C₈ to C₃₀ ester, and a mixture thereof; and an acidic buffer system. In a preferred embodiment, the vasoactive prostaglandin is prostaglandin E₁. Preferably the semi-solid composition is packaged in a unit dose and suitably the dose of the prostaglandin E₁ is about 0.05 mg to about 0.8 mg per unit dose, preferably about 0.1 mg to about 0.5 mg per unit dose. In another embodiment, the dose of the prostaglandin E₁ is about 0.1 mg to about 0.3 mg per unit dose.

A preferred penetration enhancer is an alkyl-2-(N-substituted amino)-alkanoate ester, an (N-substituted amino)-alkanol alkanoate, or a mixture of these. The buffer system provides a buffered pH value for the composition in the range of about 3 to about 7.4. A preferred pH value is about 3 to about 6.5, most preferably from about 3.5 to about 6. If desired, stabilizers, preservatives and emulsifiers may be included. In some embodiments, the composition exhibits non-Newtonian rheological properties, suitably comprising a shear-thinning polysaccharide gum or a shear-thinning polyacrylic acid polymer. In one embodiment, the composition is thixotropic. In another embodiment, the composition is pseudoplastic. In a preferred embodiment, the composition has a viscosity of about 5,000 centipoise (cps) to about 20,000 cps, more preferably from about 7,000 cps to about 13,000 cps.

A preferred pharmaceutical composition suitable for intranavicular application comprises prostaglandin E₁, a penetration enhancer, a modified polysaccharide gum, a lipophilic compound, and an acidic buffer system. The penetration enhancer is selected from the group consisting of an alkyl-2-(N-substituted amino)-alkanoate, an alkyl-2-(N,N-disubstituted amino)-alkanoate, an (N-substituted amino)-alkanol alkanoate, an (N, N-disubstituted amino)-alkanol alkanoate, pharmaceutically acceptable salts thereof and mixtures thereof. The lipophilic compound may be an aliphatic C₁ to C₈ alcohol, an aliphatic C₈ to C₃₀ ester, or a mixture of these. If desired, stabilizers, preservatives and emulsifiers may be included.

In another embodiment, the present invention provides for the use of a composition comprising prostaglandin E₁, a penetration enhancer, a shear-thinning polymer selected from the group consisting of a polysaccharide gum and a polyacrylic acid polymer, a lipophilic compound, and an acidic buffer system in the manufacture of a medicament for the treatment of erectile dysfunction in an individual suffering from at least one co-morbid condition such as diabetes mellitus, hypertension, cardiac disease, recovery from prostatectomy or erectile dysfunction unresponsive to oral phosphodiesterase-5 inhibitor therapy.

Compositions to be administered can take the form of a semi-solid suitable for intranavicular application. In use as an intranavicular agent, these compositions provide effective prostaglandin penetration into the glans penis and produce bioavailability without requiring a wasteful overloading prostaglandin concentration in the tissue. The compositions further exhibit reduced irritation, sensitivity and damage of local tissues. These pharmaceutical compositions are packaged preferably in a single dose dispenser.

Other and further aims, purposes, features, advantages, embodiments and the like will be apparent to those skilled in the art from the present specification and the appended claims.

### BRIEF DESCRIPTION OF THE DRAWINGS

In the drawings,

FIG. 1 is a diagram of the anatomical structure of the human penis in longitudinal section view;

FIG. 2 is a schematic diagram of the anatomical details of the distal portion of the human penis in longitudinal section; and

FIGS. 3A and 3B are schematic diagrams illustrating the method of administrating the topical prostaglandin E₁ composition. FIG. 3A shows the method of holding the meatus open by applying pressure to the glans on either side of the meatus, thereby spreading the meatus. FIG. 3B shows the administration of the medication dropwise through the open meatus without inserting the tip of applicator or dispenser into the meatus.

### DETAILED DESCRIPTION OF THE PREFERRED EMBODIMENTS

It has been found that that a semi-solid prostaglandin E₁ composition suitable for the treatment of erectile dysfunction in the presence of a co-morbid condition can be placed advantageously in a natural enlarged space immediately proximal to the penile meatus, the fossa navicularis. As used herein, “co-morbid” refers to a medical condition that is present in a patient who also suffers from erectile dysfunction.

The fossa navicularis provides a restricted site that is ideally suited for the application of pharmaceutical compositions. The space is lined by a non-keratinized stratified squamous epithelium and is thereby distinguished from the surface skin covering the glans and the rest of the penis and from the stratified columnar epithelium of the lining of the urethra proper. The lining of the fossa navicularis thus provides enhanced permeability compared to the keratinized epithelium of the surface skin of the outside of the penis. It has been found that the administration of the composition of the present invention in the fossa navicularis has high efficacy and low incidence of local side effects.

The fossa navicularis is a natural expanded chamber suitably adapted to receive and retain semisolid medicaments. A semi-solid medicament, such as the composition of the present invention, when placed in the fossa has higher impedance to flow at narrowed exits of this space, the meatus and the urethra. The impedance to flow is proportional to the product of the cross sectional area of the path and the path length. Thus, a semi-solid medication of suitably chosen viscosity is naturally retained within the fossa, facilitating the absorption of active agents such as vasodilators and the like. Viscosity of the composition suitably ranges from about 5,000 cps to about 20,000 cps, preferably from about 7,000 cps to about 13,000 cps.

The fossa navicularis is part of the natural defense system that protects the body against infection. The fossa navicularis is a more immunologically protected site than the adjacent pars spongiosa region of the penile urethra proper. Depositing a semisolid medicament within the anatomical limits of the fossa navicularis thus does not circumvent the natural barriers to disease by artificially transporting contaminants, e.g., from the surface of the penis, directly into the penile urethra proper. The relatively high glycogen content and bacterial flora within the fossa navicularis provides a naturally lower pH within the space, so that relatively lower pH compositions in the acidic range that provide for enhanced solubility of prostaglandin E₁ can be more easily tolerated without excessive irritation of the tissues.

Semi-solid compositions and penetration enhancers suitable for the practice of the present invention are described in detail in U.S. Pat. Nos. 6,046,244, 6,118,020 and 6,323,241, the teachings of which are incorporated herein by reference.

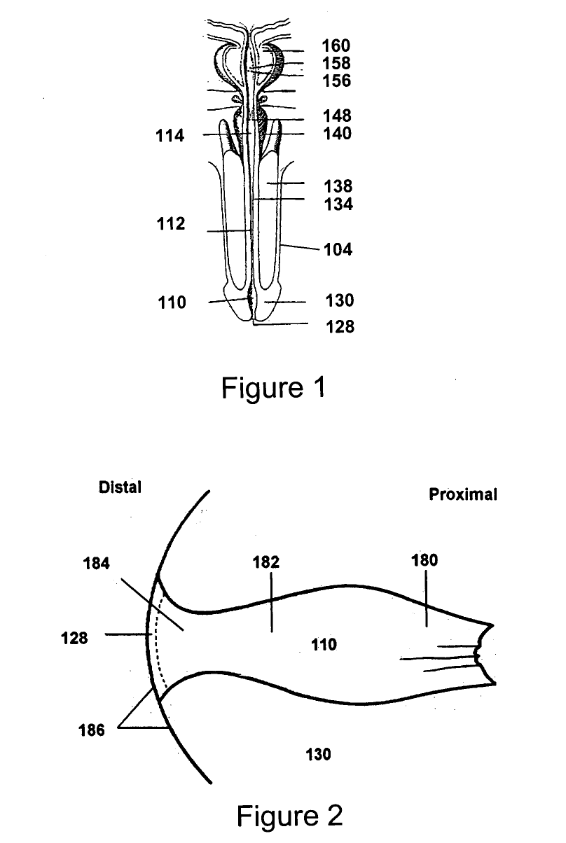

Referring to FIG. 1, the basic structure of the human penis is illustrated. The fossa navicularis 110 in the glans penis 130 is a natural enlargement of the lumen of the male urethra. The fossa navicularis extends distally to the urethral meatus 128 and proximally to the pendulous region of the urethra 112 (also termed “pars spongiosa” region of the urethra), the portion of the urethra that passes through the corpus spongiosum 134 which is found within the shaft 104 of the penis medial to the paired corpora cavernosa 138. The bulbar urethra 114 is proximal to the pendulous region of the urethra, and passes through the bulbospongiosus muscle 140. More proximally, the opening 148 in the wall of the urethra of the bulbourethral glands (Cowper's glands) can be seen. More proximally, the urethra passes through the prostate gland 160, where openings of ejaculatory duct 156 and of the prostate utricle 158 are visible in the wall of the urethra.

Referring to FIG. 2, the detailed structure of the fossa navicularis 110 within the glans penis 130 is illustrated. The external opening of the urethral meatus 128 is the distal limit of the fossa navicularis. The external skin of the glans is covered by a keratinized stratified squamous epithelium 186 (Pudney, J., and Anderson, D; J., (1995) Immunobiology of the human penile urethra, Amer. J. Path., 147: 155-165) that is marked by proximally by a sharp transition (dashed line) to the nonkeratinized stratified squamous epithelium without glycogen 184 that is characteristic of the lining of the distal fossa navicularis.

The fossa navicularis widens proximally and the lining changes to a nonkeratinized stratified squamous epithelium with glycogen 182. The glycogen in this region is believed to support a bacterial flora that lowers the pH of the region and contributes to a natural defense against infection. Holstein, A. F., et al., (1991). Different epithelia in the distal human male urethra, Cell Tiss. Res. 264: 23-32. This nonkeratinized stratified squamous epithelium with glycogen is under hormonal control, and increases in extent under increased estrogen levels (Holstein, et al., 1991). The proximalfossa navicularis narrows in width, and is lined by a stratified columnar epithelium 180.

The semi-solid composition has a suitably chosen viscosity such that the composition is naturally retained within the fossa navicularis. The semi-solid composition can exhibit Newtonian or non-Newtonian rheological characteristics. In some preferred embodiments, the semi-solid composition of the present invention exhibits non-Newtonian rheological characteristics, i.e. in which the apparent viscosity is dependent on the shear rate applied to the composition. Preferably the composition has “shear-thinning” rheological properties. As used herein, “shear-thinning” refers to a reduction in apparent viscosity (the ratio of shear stress to the shear rate) with increasing shear rate, whether the reduction in apparent viscosity is time independent (pseudoplastic), time dependent (thixotropic) or associated with a yield stress, defined as a stress that must be exceeded before flow starts, (Bingham plastics and generalized Bingham plastics). See, generally, Harris, J., & Wilkinson, W. L., “Non-newtonian Fluid,” pp.856-858 in Parker, S. P., ed., McGraw-Hill Encyclopedia of Physics, Second Edition, McGraw-Hill, New York, 1993. A suitable viscosity range of the composition is from about 5,000 centipoise (cps) to about 20,000 cps, preferably from about 7,000 cps to about 13,000 cps.

In a preferred embodiment, the pharmaceutical composition comprises at least one vasoactive prostaglandin, preferably prostaglandin E₁, an alkyl (N,N-disubstituted amino) ester, a polysaccharide gum, a lipophilic component, and an acid buffer system. The prostaglandin can be dissolved or substantially uniformly dispersed in the topical composition, preferably soluble (and dissolved) in the topical composition.

Vasoactive prostaglandins are those that act as peripheral vasodilators, including naturally occurring prostaglandins such as PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α}; semisynthetic or synthetic derivatives of natural prostaglandins, including carboprost tromethamine, dinoprost tromethamine, dinoprostone, lipoprost, gemeprost, metenoprost, sulprostone and tiaprost. Prostaglandin E₁ and prostaglandin E₂ are particularly preferred vasoactive prostaglandins for use in conjunction with the present method.

The quantity of vasoactive prostaglandin, such as prostaglandin E₁, in the pharmaceutical compositions of the present invention is a therapeutically effective (i.e., erection inducing) amount and necessarily varies according to the particular vasoactive prostaglandin to be delivered, the indication to be treated, the surface area of the skin and mucous membrane over which the formulation is to be placed, the other components of the composition, the desired dose, the dosage form (e.g., suppository or topical), and the particular form of the vasoactive prostaglandin used. The term “prostaglandin” as used generically herein refers to the prostaglandin free acid and pharmaceutically acceptable derivatives thereof, including e.g., prostaglandin E₁ (PGE₁), pharmaceutically acceptable salts and lower alkyl esters thereof (the term “lower alkyl” as used herein means straight chain or branched chain alkyl containing one to four carbon atoms). The composition generally contains about 0.001 weight percent to 1 weight percent prostaglandin E₁, typically contains about 0.05 weight percent to 1 weight percent prostaglandin E₁, preferably about 0.1 weight percent to 0.5 weight percent, based on the total weight of the composition. In one embodiment, prostaglandin E₁ is present in the composition in an amount of about 0.07 weight percent of the total composition to about 0.4 weight percent of the total composition.

Prostaglandin E₁ is well known to those skilled in the art. Accordingly, it is not practical to enumerate particular preferred amounts but such can be readily determined by those skilled in the art with due consideration of the factors listed above. Reference may be had to various literature references for its pharmacological activities, side effects, and normal dosage ranges. See for example, Physician's Desk Reference, 51 st Ed. (1997), The Merck Index, 12th Ed., Merck & Co., N.J. (1996), and Martindale The Extra Pharmacopoeia, 28th Ed., London, The Pharmaceutical Press (1982).

Additionally, simultaneous administration of one or more non-ecosanoid vasodilators may be desirable and may in some cases exhibit a synergistic effect. The combination of prazosin with prostaglandin E₁ has been found to be particularly advantageous in this regard; the latter drug appears to act as a potentiator for prazosin.

Suitable non-ecosanoid vasodilators include, but are not limited to: nitrates such as nitroglycerin, isosorbide dinitrate, erythrityl tetranitrate, amyl nitrate, sodium nitroprusside, molsidomine, linsidomine chlorhydrate (“SIN-1”) and S-nitroso-N-acetyl-d,l-penicillamine (“SNAP”); amino acids such as L-arginine; long and short acting α-adrenergic blockers such as phenoxybenzamine, dibenamine, phentolamine, tamsulosin and indoramin, especially quinazoline derivatives such as alfuzosin, bunazosin, doxazosin, terazosin, prazosin, and trimazosin; vasodilative natural herbal compositions and bioactive extracts thereof, such as gosyajinki-gan, Satureja obovata, bai-hua qian-hu, lipotab, saiboku-to, vinpocetine, Gingko biloba, bacopa, Gynostemma pentaphyllum, gypenosides, Evodia rutaecarpa, rutaecarpine, dehydroevodiamine, dan-shen, salviae miltiorrhizae radix, shosaikoto, Zizyphi fructus, ginseng and mixtures thereof (U.S. Pat. No. 6,007,824); ergot alkaloids such as ergotamine and ergotamine analogs, e.g., acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride; antihypertensive agents such as diazoxide, hydralazine and minoxidil; vasodilators such as nimodepine, pinacidil, cyclandelate, dipyridamole and isoxsuprine; chlorpromazine; haloperidol; yohimbine; trazodone and vasoactive intestinal peptides.

When used in combination with a vasoactive prostaglandin, a piperazinyl quinazoline antihypertensive, such as prazosin, is present in the amount of about 0.1 mg to about 2.0 mg per unit dose, depending on the potency of the particular piperazinyl quinazoline antihypertensive and the type and dose of vasoactive prostaglandin used. The dose and the proportion of vasoactive prostaglandin and the piperazinyl quinazoline antihypertensive can be routinely determined by one of ordinary skill without undo experimentation.

The topical composition can contain one or more penetration enhancers. Among the preferred penetration enhancers for the present invention are ethanol, propylene glycol, glycerol, ethyl laurate, isopropyl palmitate, isopropyl myristate, laurocapram (Azone™), dioxolanes (described in U.S. Pat. No. 4,861,764), macrocyclic ketones, HP-101, oxazolidones and biodegradable penetration enhancers (described in U.S. Pat. Nos. 4,980,378 and 5,082,866 to Wong et al. and U.S. Pat. No. 6,118,020 to Büyüktimkin et al. such as alkyl-2-(N-substituted amino) alkanoates, alkyl-2-(N,N-disubstituted amino) alkanoates (e.g., dodecyl N,N-dimethylamino isoproprionate (DDAIP)), N-substituted amino alkanol alkanoates), N,N-disubstituted amino alkanol alkanoates, acid addition salts and mixtures thereof. For example, the preparation of crystalline acid addition salts of DDAMP by cooled mixing of DDAIP with one of a select group of acids in the presence of a water-immiscible solvent such as hexane, is disclosed in U.S. Pat. No. 6,118,020, the contents of which are incorporated herein by reference in their entirety. Acid addition salts of dodecyl 2-(N,N-dimethylamino)-propionate (DDAIP) can be inorganic as well as organic. Representative inorganic acid addition salts include the hydrochloric, hydrobromic, sulfuric, phosphoric, nitric acid addition salts of DDAIP, and their solvates. Exemplary organic acid addition salts include acetic, benzoic, salicylic, glycolic, succinic, nicotinic, tartaric, maleic, malic, palmoic, methanesulfonic, cyclohexanesulfamic, picric, and lactic acid addition salts, as well as their respective solvates. Preferred among the inorganic acid addition salts are DDAIP hydrogen chloride, and DDAIP dihydrogen sulfate.

The penetration enhancer is present in an amount sufficient to enhance the penetration of the prostaglandin E₁. The specific amount varies necessarily according to the desired release rate and the specific form of prostaglandin E₁ used. Generally, the penetration enhancer is present in an amount ranging from about 0.5 weight percent to about 20 weight percent, based on the total weight of the composition. Preferably, the penetration enhancer is present in an amount ranging from about 1 weight percent to about 10 weight percent of the composition. More preferably, the penetration enhancer is present in an amount ranging from about 1 weight percent to about 5 weight percent of the composition.

In general, suitable penetration enhancers can be chosen from those listed above as well as sulfoxides, alcohols, fatty acids, fatty acid esters, polyols, amides, surfactants, terpenes, alkanones, organic acids and mixtures thereof. See generally Chattaraj, S. C. and Walker, R. B., Penetration Enhancer Classification, pp.5-20 in Maibach, H. I., and Smith, H. E., (eds.), Percutaneous Penetration Enhancers, CRC Press, Inc., Boca Raton, Fla. (1995) and Büyüktimkin, N., et al., Chemical Means of Transdermal Drug Permeation Enhancement, in Gosh, T. K., et al., (eds.) Transdermal and Topical Drug Delivery Systems, Interpharm Press, Inc., Buffalo Grove, Ill. (1997). Suitable sulfoxides include dimethylsulfoxide, decylmethylsulfoxide and mixtures thereof. Suitable alcohols include ethanol, propanol, butanol, pentanol, hexanol, octanol, nonanol, decanol, 2-butanol, 2-pentanol, benzyl alcohol, caprylic alcohol, decyl alcohol, lauryl alcohol, 2-lauryl alcohol, myristyl alcohol, cetyl alcohol, stearyl alcohol, olcyl alcohol, linolyl alcohol, linolenyl alcohol and mixtures thereof. Suitable fatty acids include valeric, heptanoic, pelargonic, caproic, capric, lauric, myristic, stearic, oleic, linoteic, linolenic, caprylic, isovaleric, neopentanoic, neoheptanoic, neononanoic, trimethyl hexanoic, neodecanoic and isostearic acids and mixtures thereof.

Suitable fatty acid esters include isopropyl n-butyrate, isopropyl n-hexanoate, isopropyl n-decanoate, isopropyl myristate, isopropyl palmitate, octyldodecyl myristate, ethyl acetate, butyl acetate, methyl acetate, methylvalerate, methylpropionate, diethyl sebacate, ethyl oleate, ethyl laurate and mixtures thereof. Suitable polyols include propylene glycol, polyethylene glycol, ethylene glycol, diethylene glycol, triethylene glycol, dipropylene glycol, glycerol, propanediol, sorbitol, dextrans, butanediol, pentanediol, hexanetriol and mixtures thereof.

Suitable amides include urea, dimethylacetamide, diethyltoluamide, dimethylformamide, dimethyloctamide, dimethyldecamide, 1-alkyl-4-imidazolin-2-one, pyrrolidone derivatives, cyclic amides, hexamethylenelauramide and its derivatives, diethanolamine, triethanolamine and mixtures thereof. Suitable pyrrolidone derivatives include 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-decyl-thioethyl-2-pyrrolidone (HP-101), 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-cyclohexylpyrrolidone, N-dimethylaminopropylpyrrolidone, N-cocoalkypyrrolidone, N-tallowalkypyrrolidone, fatty acid esters of N-(2-hydroxymethyl)-2-pyrrolidone and mixtures thereof. Suitable cyclic amides include 1-dodecylazacycloheptane-2-one (laurocapram, Azone™), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyloctyl)azacycloheptan-2-one, 1-geranylazacyclohexane-2-one, 1-geranylazacyclopentan-2,5-dione, 1-farnesylazacyclopentan-2-one and mixtures thereof.

Suitable surfactants include anionic surfactants, cationic surfactants, nonionic surfactants, bile salts and lecithin. Suitable anionic surfactants include sodium laurate, sodium lauryl sulfate and mixtures thereof. Suitable cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride, and mixtures thereof. Suitable nonionic surfactants include α-hydro-ω-hydroxy-poly(oxyethylene)-poly(oxypropyl) poly(oxyethylene)block copolymers, polyoxyethylene ethers, polyoxyethylene sorbitan esters, polyethylene glycol esters of fatty alcohols and mixtures thereof. Suitable α-hydro-ω-hydroxy-poly(oxyethylene)-poly(oxypropyl) poly(oxyethylene)block copolymers include Poloxamers 231, 182, and 184 and mixtures thereof. Suitable polyoxyethylene ethers include 4-lauryl ether (BRIJ 30™), (BRIJ 93™), (BRIJ 96™), 20-oleyl ether (BRIJ 99™) and mixtures thereof. Suitable polyoxyethylene sorbitan esters include the monolaurate (TWEEN 20™, SPAN 20™) the monopalmitate (TWEEN 40™), the monostearate (TWEEN 60™), and the monooleate (TWEEN 80™) and mixtures thereof. Suitable polyethylene glycol esters of fatty acids include the 8-oxyethylene stearate ester (MYRJ 45™), (MYRJ 51™), the 40-oxyethylene stearate ester (MYRJ 52™) and mixtures thereof. Suitable bile salts include sodium cholate, sodium salts of laurocholic, glycolic and desoxycholic acids and mixtures thereof.

Suitable terpenes include D-limonene, α-pinene, β-enrene, α-terpineol, terpinen-4-ol, carvol, carvone, pulegone, piperitone, menthone, menthol, geraniol, cyclohexene oxide, limonene oxide, α-pinene oxide, cyclopentene oxide, 1,8-cineole, ylang ylang oil, anise oil, chenopodium oil, eucalyptus oil and mixtures thereof. Suitable alkanones include N-heptane, N-octane, N-nonane, N-decane, N-undecane, N-dodecane, N-tridecane, N-tetradecane, N-hexadecane and mixtures thereof. Suitable organic acids include citric acid, succinic acid, salicylic acid, salicylates (including the methyl, ethyl and propyl glycol derivatives), tartaric acid and mixtures thereof.

Natural and modified polysaccharide gums are also an important ingredient of the composition. Suitable representative gums are those in the natural and modified galactomannan gum category. A galactomannan gum is a carbohydrate polymer containing D-galactose and D-mannose units, or other derivatives of such a polymer. There is a relatively large number of galactomannans, which vary in composition depending on their origin. The galactomannan gum is characterized by a linear structure of β-D-mannopyranosyl units linked (1→4). Single membered α-D-manopyranosyl units, linked (1→6) with the main chain, are present as side branches. Galactomannan gums include guar gum, which is the pulverized endosperm of the seed of either of two leguminous plants (Cyamposis tetragonalobus and psoraloids) and locust bean gum, which is found in the endosperm of the seeds of the carobtree (ceratonia siliqua). Suitable modified polysaccharide gums include ethers of natural or substituted polysaccharide gums, such as carboxymethyl ethers, ethylene glycol ethers and propylene glycol ethers. An exemplary substituted polysaccharide gum is methylcellulose. A preferred modified galactomannan gum is modified guar gum.

Other suitable representative gums include agar gum, carrageenan gum, ghatti gum, karaya gum, rhamsan gum and xanthan gum. The composition of the present invention may contain a mixture of various gums, or mixture of gums and acidic polymers.

Gums, and galactomannan gums in particular, are well-known materials. See for instance, Industrial Gums: Polysaccharides & Their Derivatives, Whistler R. L. and BeMiller J. N. (eds.), 3rd Ed. Academic Press (1992) and Davidson R. L., Handbook of Water-Soluble Gums & Resins, McGraw-Hill, Inc., N.Y. (1980). Most gums are commercially available in various forms, commonly a powder, and ready for use in foods and topical compositions. For example, locust bean gum in powdered form is available from Tic Gums Inc. (Belcam, Md.).

When present, the polysaccharide gums are present in the range from about 0.1 percent to about 5 percent, based on the total weight of the composition, with the preferred range being from 0.5 percent to 3 percent. In one preferred embodiment, 2.5 percent by weight of a polysaccharide gum is present. Illustrative compositions are given in the examples, below.

An optional alternative to the polysaccharide gum is a polyacrylic acid polymer. A common variety of polyacrylic acid polymer is known generically as “carbomer.” Carbomer is polyacrylic acid polymers lightly cross-linked with polyalkenyl polyether. It is commercially available from the B. F. Goodrich Company (Akron, Ohio) under the designation “CARBOPOL™.” A particularly preferred variety of carbomer is that designated as “CARBOPOL 940.”

Other polyacrylic acid polymers suitable for use are those commercially available under the designations “Pemulen™” (B. F. Goodrich Company) and “POLYCARBOPHIL™” (A. H. Robbins, Richmond, Va.). The Pemulen™ polymers are copolymers of C₁₀ to C₃₀ alkyl acrylates and one or more monomers of acrylic acid, methacrylic acid or one of their simple esters crosslinked with an allyl ether of sucrose or an allyl ether of pentaerythritol. The POLYCARBOPHIL™ enhancer is a polyacrylic acid cross-linked with divinyl glycol.

Where polyacrylic acid polymers are present, they represent about 0.5 percent to about 5 percent of the composition, based on its total weight.

Another important component is a lipophilic component. As used herein “lipophilic component” refers to an agent that is both lipophilic and hydrophilic. One of ordinary skill in the pharmaceutical arts will understand that the lipophilic nature, or “lipophilicity” of a given compound is routinely quantified for comparison to other compounds by using the partition coefficient. The partition coefficient is defined by the International Union of Pure and Applied Chemistry (IUPAC) as the ratio of the distribution of a substance between two phases when the heterogeneous system (of two phases) is in equilibrium; the ratio of concentrations (or, strictly speaking, activities) of the same molecular species in the two phases is constant at constant temperature.

The C₁ to C₈ aliphatic alcohols, the C₂ to C₃₀ aliphatic esters, and their mixtures can serve as lipophilic component. Illustrative suitable alcohols are ethanol, n-propanol and isopropanol, while suitable esters are ethyl acetate, butyl acetate, ethyl laurate, methyl propionate, isopropyl myristate and isopropyl palmitate. As used herein, the term “aliphatic alcohol” includes polyols such as glycerol, propylene glycol and polyethylene glycols. In one embodiment, a mixture of alcohol and ester is preferred, and in particular, a mixture of ethanol and ethyl laurate is preferred.

In one embodiment, the C₂ to C₃₀ aliphatic esters, and their mixtures comprising the lipophilic component include C₈ to C₃₀ aliphatic esters of glycerol selected from the group consisting monoglycerides, diglycerides, triglycerides, and mixtures thereof. Suitable aliphatic esters include glyceryl esters of saturated fatty acids, unsaturated fatty acids and mixtures thereof. Suitable saturated fatty acids include caproic acid, caprylic acid, capric acid, lauric acid, myristic acid, palmitic acid, stearic acid, arachidic acid, behenic acid and lignoceric acid. Suitable unsaturated fatty acids include oleic acid, linoleic acid and linolenic acid. Suitable glyceryl esters include glyceryl monooleate, triolein, trimyristin and tristearin, perferably trimyristin.

The concentration of lipophilic component required necessarily varies according to other factors such as the desired semi-solid consistency and the desired skin penetration promoting effects. Suitably the concentration of lipophilic component is in the range of 0.5 percent to 40 percent by weight based on the total weight of the composition. The preferred topical composition contains lipophilic component in the range of 7 percent to 40 percent by weight based on the total weight of the composition.

Where a mixture of aliphatic alcohol and aliphatic ester are employed, the suitable amount of alcohol is in the range of 0.5 percent to 10 percent. In one preferred embodiment, the amount of alcohol is in the range of 5 percent to 15 percent, while that of aliphatic ester is in the range from 2 percent to 15 percent (again based on the total weight of the composition). In another preferred embodiment, the amount of alcohol is in the range of 0.5 percent to 10 percent, while that of aliphatic ester is in the range from 0 percent to 10 percent (again based on the total weight of the composition).

The concentration of lipophilic component required necessarily varies according to other factors such as the desired semi-solid consistency and the desired skin penetration promoting effects. The preferred topical composition contains lipophilic component in the range of 7 percent to 40 percent by weight based on the total weight of the composition. Where a lipophilic component that is a mixture of aliphatic alcohol and aliphatic ester is used, the preferred amount of alcohol is in the range of 5 percent to 15 percent, while that of aliphatic ester is in the range from 2 percent to 15 percent (again based on the total weight of the composition).

An optional, but preferred, component is an emulsifier. Although not a critical factor, a suitable emulsifier generally will exhibit a hydrophilic-lipophilic balance number greater than 10. Sucrose esters, and specifically sucrose stearate, can serve as emulsifiers for the composition. Sucrose stearate is a well-known emulsifier available from various commercial sources. When an emulsifier is used, sucrose stearate present up to about 2 percent, based on the total weight of the composition, is preferred. The preferred amount of sucrose stearate emulsifier can also be expressed as a weight ratio of emulsifier to polysaccharide gum. A ratio of 1 to 6 emulsifier to gum is preferred, and a ratio of 1 to 4 is most preferred to generate the desired semi-solid consistency and separation resistance.

Other emulsifiers are also suitable including polyoxyethylene sorbitan esters, long chain alcohols, preferably cetostearyl alcohol, and fatty acid glycerides. Suitable polyoxyethylene sorbitan esters include the monolaurate (Tween 20™, Span 20™) the monopalmitate (Tween 40™), the monostearate (Tween 60™), and the monooleate (Tween 80™) and mixtures thereof. Preferred fatty acid glycerides include glyceryl monooleate, triolein, trimyristin and tristearin.

The composition includes an acid buffer system. Acid buffer systems serve to maintain or buffer the pH of compositions within a desired range. The term “buffer system” or “buffer” as used herein has reference to a solute agent or agents which, when in a water solution, stabilize such solution against a major change in pH (or hydrogen ion concentration or activity) when acids or bases are added thereto. Solute agent or agents which are thus responsible for a resistance to change in pH from a starting buffered pH value in the range indicated above are well known. While there are countless suitable buffers, potassium phosphate monohydrate has proven effective for compositions of the present invention.

The final pH value of the pharmaceutical composition may vary within the physiologically compatible range. Necessarily, the final pH value is not irritating to human skin. Without violating this constraint, the pH may be selected to improve prostaglandin E₁ stability and to adjust consistency when required. In one embodiment, the preferred pH value is about 3 to about 7.4, more preferably about 3 to about 6.5, most preferably from about 3.5 to about 6.

The remaining component of the composition is water, which is necessarily purified. The composition contains water in the range of about 50 to about 90 percent, based on the total weight of the composition. The specific amount of water present is not critical, however, being adjustable to obtain the desired consistency and/or concentration of the other components.

Prostaglandin E₁ stabilizers, coloring agents, Theological agents, and preservatives can be added to the extent that they do not overly limit prostaglandin E₁ skin penetration or prevent the desired semi-solid consistency.

Contemplated dosage forms of the semi-solid pharmaceutical composition are creams, gels, ointments, colloidal suspensions and the like, also including but not limited to compositions suitable for use with transdermal patches and like devices.

The ingredients listed above may be combined in any order and manner that produces a stable composition comprising a prostaglandin E₁ evenly dispersed throughout a semi-solid formulation. One available approach to preparing such compositions involves evenly dispersing the polysaccharide gum (or polyacrylic acid polymer) in a premixed water/buffer solution and then thoroughly homogenizing (i.e. mixing) the resulting mixture, which will be referred to as “Part A.” When present, the emulsifier is added to the water/buffer solution before dispersing the polysaccharide gum. Any suitable method of adjusting the pH value of Part A to the desired level may be used, for example, by adding concentrated phosphoric acid or sodium hydroxide.

Separately, the prostaglandin E₁ is dissolved with agitation in the lipophilic component, which itself may be a mixture of alcohols, esters, or alcohol with ester. Next, the penetration enhancer is added. Alternatively, when the lipophilic component includes both an alcohol and an ester, the prostaglandin E₁ can be dissolved in the alcohol before adding the penetration enhancer followed by the ester. In either case, the resulting mixture will be referred to as “Part B.” The final step involves slow addition (e.g. dropwise) of Part B into Part A under constant mixing.

The resulting topical composition, when compared to exhibits the advantageous properties described above, including improved prostaglandin E₁ permeation and bioavailability without drug overloading, reduced skin damage and related inflammation, and increased flexibility in design of dosage forms. These compositions can be used for prolonged treatment of peripheral vascular disease, male impotency and other disorders treated by prostaglandin E₁, while avoiding the low bioavailability and rapid chemical decomposition associated with other delivery methods. Application of prostaglandin E₁ in a topical composition to the skin of a patient allows a predetermined amount of prostaglandin E₁ to be administered continuously to the patient and avoids undesirable effects present with a single or multiple administrations of larger dosages by injection. By maintaining a sustained dosage rate, the prostaglandin E₁ level in the patient's target tissue can be better maintained within the optimal therapeutic range.

In one embodiment, a composition comprises about 0.01 percent to about 5 percent modified polysaccharide gum; about 0.001 percent to about 1 percent of a prostaglandin selected from the group consisting of PGE₁, pharmaceutically acceptable salts thereof, lower alkyl esters thereof and mixtures thereof; about 0.5 percent to about 10 percent DDAIP or salts thereof; about 0.5 percent to about 10 percent of a lower alcohol selected from the group consisting of ethanol, propanol, isopropanol and mixtures thereof; about 0.5 percent to about 10 percent on an ester selected from the group consisting of ethyl laurate, isopropyl myristate, isopropyl laurate and mixtures thereof; based on the weight of the composition, and an acid buffer. Preferably the composition also comprises up to about 2 percent sucrose stearate.

Optionally the composition also comprises up to about 5 percent emulsifier. Preferably, the composition also comprises up to about 2 percent emulsifier. Suitable emulsifiers include polysorbates such as Tweens, glyceryl monooleate, triolein, trimyristin and tristearin. A preferred emulsifier is trimyristin.

The practice of the present invention is demonstrated in the following examples. These examples are meant to illustrate the invention rather than to limit its scope. Variations in the treating compositions which do not adversely affect the effectiveness of prostaglandin E₁ will be evident to one skilled in the art, and are within the scope of this invention. For example, additional ingredients such as coloring agents, anti-microbial preservatives, emulsifiers, perfumes, prostaglandin E₁ stabilizers, and the like may be included in the compositions as long as the resulting composition retains desirable properties, as described above. When present, preservatives are usually added in amounts of about 0.05 to about 0.30%. Suitable preservatives include methylparabens (methyl PABA), propylparabens (propyl PABA) and butylhydroxy toluene (BHT). Suitable perfumes and fragrances are known in the art; a suitable fragrance is up to about 5 percent myrtenol, preferably about 2 percent myrtenol, based on the total weight of the composition. The compositions of the present invention can also include a small amount, about 0.01 to about 4% by weight, of a topical anesthetic, if desired. Typical topical anesthetics include lidocaine, dyclonine, dibucaine, pharmaceutically acceptable salts and mixtures thereof. In one preferred embodiment, the topical anesthetic is about 0.5 percent dyclonine, based on the weight of the composition.

The pharmaceutical preparation is preferably in unit dosage form. In such form the preparation is subdivided into unit doses containing appropriate quantities of the active component. The unit dosage form is a packaged preparation, where the package containing the discrete quantities of the pharmaceutical preparation is, e.g. a rigid plastic dispenser or flexible packet.

Another aspect of the invention is an article of manufacture that comprises a composition for treating erectile dysfunction as described above in a suitable container, preferably in a container such as the dispenser disclosed in U.S. Pat. No. 6,224,573, in combination with labeling instructions. Alternatively, the container can be a tube with a suitable orifice size, such as an extended tip tube, pouch, packet, or squeeze bottle and made of any suitable material, for example rigid plastic or flexible plastic.

The labeling instructions can come in the form of a pamphlet, a label applied to or associated with the packaging of the article of manufacture.

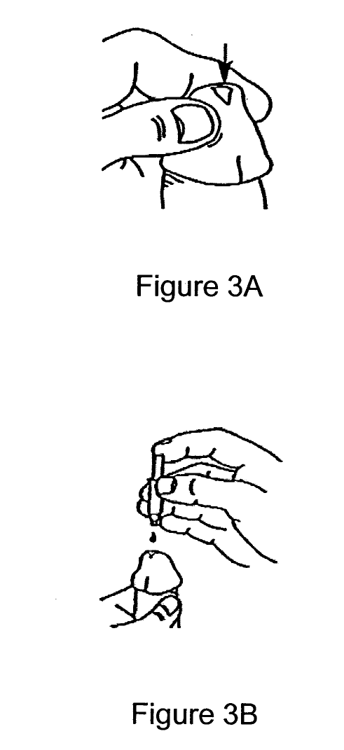

The labeling instructions provide for administering a composition of the invention to the fossa navicularis of the penis of a patient suffering from erectile dysfunction, directing the patient to hold the penis upright, hold the meatus open and place the composition in the fossa navicularis without introducing the tip of the container into the meatus, about 5-30 minutes before sexual intercourse, see FIGS. 3A-3B. Printed labeling instructions are functionally related to the composition of the invention inasmuch as such labeling instructions describe a method to treat erectile dysfunction according to the present invention. The labeling instructions are an important aspect of the invention in that before a composition can be approved for any particular use, it must be approved for marketing by the responsible national regulatory agency, such as the United States Food and Drug Administration. Part of that process includes providing a label that will accompany the pharmaceutical composition which is ultimately sold. While the label will include a definition of the composition and such other items such as the clinical pharmacology, mechanism of action, drug resistance, pharmacokinetics, absorption, bioavailability, contraindications and the like, it will also provide the necessary dosage, administration and usage. Thus, the combination of the composition with the dispenser with appropriate treatment instructions is important for the proper usage of the drug once it is marketed to the patient. Such treatment instructions will describe the usage in accordance with the method of treatment set forth herein before.

The quantity of active component in a unit dose preparation may be varied or adjusted from 0.01 mg to 1 g according to the particular application and the potency of the vasoactive prostaglandin. For example, where the vasoactive prostaglandin is prostaglandin E₁, about 0.05 mg to about 0.8 mg prostaglandin E₁ is present per unit dose, preferably about 0.1 mg to about 0.5 mg per unit dose and in another embodiment, about 0.1 mg to about 0.3 mg per unit dose. The composition can, if desired, also contain other compatible therapeutic agents, such as a piperazinyl quinazoline antihypertensive.

For “on demand” treatment, the semi-solid vasoactive prostaglandin composition should be applied to the fossa navicularis of the penis about 2-30 minutes before sexual intercourse, preferably about 5-15 minutes before sexual intercourse. In some embodiments, a regular regimen of treatment not necessarily linked to anticipated sessions of sexual intercourse can be undertaken. In such embodiments, the semi-solid vasoactive prostaglandin composition can be applied to the fossa navicularis of the penis at least twice a week, preferably every other day, or on a daily basis.

Unless otherwise indicated, each composition is prepared by conventionally admixing the respective indicated components together.

### EXAMPLE 1

### Topical Prostaglandin E₁ Composition A

Composition A was prepared as follows. Part A of the composition was formed by dissolving 0.4 parts by weight prostaglandin E₁ (Alprostadil USP) in 5 parts by weight ethyl alcohol. Next, 5 parts by weight dodecyl 2-(N,N-dimethylamino)-propionate were mixed into the alcohol-prostaglandin E₁ solution, followed by 5 parts by weight ethyl laurate.

Part B was prepared starting from a pH 5.5 water/buffer solution. The water/buffer solution was prepared by adding sufficient potassium phosphate monohydride to purified water to create a 0.1 M solution. The pH of the water/buffer solution was adjusted to 5.5 with a strong base solution (1 N sodium hydroxide) and a strong acid (1 N phosphoric acid). The buffer solution represented about 80 parts of the total composition. All parts specified herein are parts by weight.

Ethyl laurate, 0.5 parts by weight, was added to the buffer solution. Next, the locust bean gum (in powder form) was dispersed in the buffer solution and homogenized using a homogenizer. Table 1, below, contains a list of the ingredients.

The resulting composition was a spreadable, semi-solid preparation suitable for application to the skin without the need for supporting devices such as patches and adhesive strips. The composition was both homogenous in appearance and resistant to separation.

Additional exemplary compositions B-H were prepared in the same manner using the components listed in Table 1. As noted above, in other embodiments, such as Composition H, the composition may include a modified polysaccharide gum, suitably a modified galactomannan gum, such as a guar gum. Alternatively, a polyacrylic polymer may be used instead of the polysaccharide gum.

Composition A was evaluated for skin penetration using shed snake skin as a model barrier. Shed snake skin was obtained from the Animal Care Unit of the University of Kansas. With head and tail sections removed, the skin was randomly divided into test sections and then hydrated by soaking.

The samples were then evaluated using Franz-type Diffusion Cells (surface area 1.8 cm²). Specifically, skin pieces were mounted on top of a receptor cell of a vertical diffusion cell assembly in which a small magnetic bar was inserted and filled with an isotonic buffer. A seal was placed on top of the skin section followed by a donor cell. The two cells were clamped together. Known amounts of the formulations were applied on the bottom of a small capped vial (weight 0.5 grams) which fits exactly to the donor cell to ensure uniform distribution. The vials were placed on the skin in the donor cell. To reduce the evaporation of the ingredients, the donor cell and vial were taped together with a water-resistant adhesive band. The cells were transferred to a stirred water bath (32 degrees Celsius). Samples were withdrawn from the cells each hour for four hours and analyzed for the concentration of prostaglandin E₁, with changes in concentration indicating the amount penetrating. Tests with multiple skin samples yielded data that were averaged. For a discussion of the use of shed snake skin in the evaluation of drug penetration, see U.S. Pat. No. 4,771,004 to Higuchi, which is incorporated here by reference.

The prostaglandin E₁ penetrated quickly at a relatively sustained rate for four hours. The results of the penetration study are presented in Table 2, below.

### EXAMPLE 2

### Topical Prostaglandin E₁ Composition B

Composition B was prepared using the ingredients listed in Table 1, below. Composition B contained more prostaglandin E₁ than Composition A. Despite this increased drug loading, Composition B exhibited a similar semi-solid consistency and homogenous appearance. The penetration of prostaglandin E₁ was measured according to the technique described in Example 1. Composition B provided a relatively fast, sustained delivery of prostaglandin E₁. The results are presented in Table 2.

### EXAMPLE 3

### Topical Prostaglandin E₁ Composition C

Composition C was prepared using the ingredients listed in Table 1, below. Composition B contained more prostaglandin E₁ than either Composition A or B. The increased drug loading had little or no effect on the consistency or appearance, which substantially matched that of Compositions A and B. The penetration of prostaglandin E₁ was again measured according to the technique described in Example 1. According to this test, Composition C also provided a relatively fast, sustained delivery of prostaglandin E₁. The results are presented in Table 2, below.

### EXAMPLE 4

### Topical Prostaglandin E₁ Composition D

Composition D was prepared using the ingredients listed in Table 1, below. The level of prostaglandin E₁ was again increased without substantially affecting the favorable consistency and separation resistance. The penetration of prostaglandin E₁ was again measured according to the technique described in Example 1. The results are presented in Table 2, below.

### EXAMPLE 5

### Topical Prostaglandin E₁ Composition E

Composition E was prepared using the ingredients listed in Table 1, below. To assess the repeatability of compositions according to the present invention, the recipe of Composition D was again applied for Composition E. Repeatability was substantially confirmed by Composition E's favorable, semi-solid consistency and separation resistance. The penetration of prostaglandin E₁ was again measured according to the technique described in Example 1. The prostaglandin E₁ delivery from Composition E was again relatively fast and sustained. The results are presented in Table 2, below.

### EXAMPLE 6

### Topical Prostaglandin E₁ Composition F

The level of prostaglandin E₁ was again increased for Composition F. The specific ingredients are listed in Table 1. The favorable consistency and separation resistance was undiminished. The results of a penetration analysis are presented in Table 2, below.

### EXAMPLE 7

### Topical Prostaglandin E₁ Composition G

Composition G was prepared using the ingredients listed in Table 1. For Composition G, the recipe of Composition F was repeated except that the ester component (ethyl laurate) was omitted and the level of ethanol was increased a corresponding amount. The resulting composition was also a spreadable, semi-solid having a homogenous appearance and resistance to separation. The results of a penetration analysis are presented in Table 2, below. While still favorable, these results reflect the relative benefit to compositions of the present invention from a lipophilic compound that includes both an ester component and an alcohol component.

TABLE 1
Topical Prostaglandin E₁ Compositions

| Ingredient (wt %) | A | B | C | D | E | F | G | H | I |
|---|---|---|---|---|---|---|---|---|---|
| prehydrated locust bean gum | 3 | 3 | 3 | 3 | 3 | 3 | 3 | — | — |
| prehydrated modified guar gum | — | — | — | — | — | — | — | 3 | 2.5 |
| water/buffer (pH 5.5) | 81 | 81 | 81 | 81 | 81 | 81 | 81 | 81 | 86.8 |
| sucrose stearate | 0.5 | 0.5 | 0.5 | 0.5 | 0.5 | 0.5 | 0.5 | — | — |
| prostaglandin E₁ | 0.1 | 0.2 | 0.3 | 0.4 | 0.4 | 0.5 | 0.4 | 0.3 | 0.2 |
| DDAIP | 5 | 5 | 5 | 5 | 5 | 5 | 5 | 2.5 | — |
| DDAIP HCl | — | — | — | — | — | — | — | — | 2.5 |
| ethanol | 5 | 5 | 5 | 5 | 5 | 5 | 10 | 5 | 5 |
| ethyl laurate | 5 | 5 | 5 | 5 | 5 | 5 | — | 3 | 3 |

### EXAMPLE 8

### Comparison of Penetration Profiles

Table 2 shows the cumulative amount of prostaglandin E₁ penetrating each hour for 4 hours for each example composition according to the present invention. These data demonstrate the ability of the present invention to deliver prostaglandin E₁ drugs transdermally.

TABLE 2
Cumulative Prostaglandin E₁ Penetration (μg/cm²)

| Hour | A | B | C | D | E | F | G |
|---|---|---|---|---|---|---|---|
| 1 | 1.96 | 3.37 | 5.47 | 7.20 | 7.09 | 10.38 | 3.03 |
| 2 | 5.49 | 9.72 | 18.06 | 21.26 | 16.6 | 25.03 | 8.17 |
| 3 | 11.25 | 18.18 | 30.34 | 35.53 | 28.24 | 42.18 | 12.93 |
| 4 | 13.98 | 23.48 | 38.49 | 47.98 | 41.1 | 52.13 | 18.71 |

To further assess the effectiveness of compositions according the present invention, comparative example compositions were prepared. A first comparative example (Comparative Example 1) was prepared with the same recipe as Compositions D and E except that the DDAIP penetration enhancer was omitted. For a second comparative example (Comparative Example 2), the DDAIP was again omitted, but the level of ethanol was increased a corresponding amount. The specific ingredients used are listed in Table 3, below.

TABLE 3
Comparative Examples

| | Comparative | Comparative |
|---|---|---|
| Ingredient (parts by weight) | Composition 1 | Composition 2 |
| prehydrated locust bean gum | 3 | 3 |
| water/buffer (pH 5.5) | 86 | 81 |
| sucrose stearate | 0.5 | 0.5 |
| prostaglandin E₁ | 0.4 | 0.4 |
| Ethanol | 5 | 10 |
| ethyl laurate | 5 | 5 |

The penetration of prostaglandin E₁ was evaluated according to the technique described in Example 1. The results are presented in Table 4, below.

TABLE 4
Comparative Examples
Cumulative Prostaglandin E₁ Penetration (μg/cm²)

| | Comparative | Comparative |
|---|---|---|
| Hour | Composition 1 | Composition 2 |
| 1 | 2.64 | 1.55 |
| 2 | 4.46 | 3.69 |
| 3 | 6.59 | 6.63 |
| 4 | 9.67 | 11.05 |

### EXAMPLE 9

### Single Use Double Blind and Open Label Clinical Trials

The safety and efficacy of a 0.4% weight prostaglandin E₁ (prostaglandin E₁ or alprostadil) topical composition (composition D of Example 4 and Table 1, above) was evaluated in a total of 143 men at three study sites. This study consisted of a double-blind, placebo controlled and cross-over portion and an open-label portion.

The double-blind placebo controlled portion of the study was entered and completed by 64 men (Table 5, below). Seventy-nine (79) men entered and completed the open-label portion of the study (Table 5, below). Summarized below are discussions on the results of the clinical studies.

Inclusion Criteria

1. Males, ages 21-70 years, inclusive.

2. Documented history of erectile dysfunction, defined as the inability to achieve and maintain an erection of sufficient rigidity for sexual intercourse due to psychogenic, neurogenic or vasculogenic causes during the previous 6 months. This includes patients who may still have some erections sufficient for intercourse but not consistently, which is the typical complaint of the age onset, mild to moderate impotent man. The diagnosis of erectile dysfunction was based on medical history and physical examination.

Exclusion Criteria

1. History of urethral stricture or obstruction.

2. Any combination of findings from history, physical examination or screening studies which indicate pre-existing impairment of heart, liver and/or kidney function (such as congestive heart failure, unstable angina or recent acute myocardial infarction, uncontrolled diabetes, for erectile dysfunction of hormonal origin) which in the investigator's opinion could influence the outcome of the study.

3. History of penile surgery, including penile implant, prostatectomy or cancer of the prostate, penile trauma including paraplegia or quadriplegia.

4. Any condition which might predispose towards priapism, such as sickle cell anemia, multiple myeloma, or leukemia.

5. Hypertension, (sitting diastolic pressure >90 or systolic >150) requiring treatment with other than angiotensin converting enzyme inhibitors (ACE inhibitors).

6. Presence of a sexually transmitted disease as determined by physical examination.

7. Use of a cavernosal injection or external erectile device within 4 weeks prior to entering into this study.

8. Peyronie's Disease or any palpable fibrous scar or plaque on the penis, evidence of curvature during tumescence and rigidity stimulation or an anomaly of the penis skin or mucosa of the glans.

9. Any concomitant medication which are known to interfere with sexual activity such as antidepressants, some antihypertensives, sedatives hormones and some allergy medications.

10. Received any investigational treatment within 30 days of entering into this study.

11. Inability or unwillingness to give informed consent.

The patient population in this study consisted of men in the age range of 49-70 years old.

TABLE 5
Patient Enrollment by Study Sites

| | Patients Enrolled On Study | | | |
|---|---|---|---|---|
| | Sites | | | |
| Portion | No. 1 | No. 2 | No. 3 | Total |
| Double-Blind | 30 | 34 | 0 | 64 |
| Open Label | 32 | 8 | 39 | 79 |

Clinical efficacy was evaluated from patient history and patient evaluation questionnaires both before and after medication using a six-point classification scale (Table 6). Each patient was given one (1) placebo and one (1) active dose in a crossover manner with a 5 to 7 day wash-off period in the double-blind portion of the study. In the open-label portion the patients were given only one (1) active dose. The clinical supply was packaged in single-dose containers each containing 250 mg (net weight) of cream and 1 mg prostaglandin E₁.

The efficacy response rate was determined as the number of men that had erections sufficient for intercourse out of the total number of men. To be considered a success, a score of 8 to 10 must be achieved after administration of the dose or the patient must have had intercourse.

Statistical analysis compared before and after response scores using a paired t-test. A statistically significant difference (P<0.001) between all before and after dosing scores was found for each group of patients receiving active medication whether in the double-blind portion of the study or the open label portion of the study. Also, a statistical significance was seen between the active and placebo groups per study site.

TABLE 6
Six-Point Classification Scale for Assessing the Severity
of Male Erectile Dysfunction (Impotence)

| Classification | Definition |
|---|---|
| 0 | Severe impotence with no function |
| 2 | Severe impotence with very little function |
| 4 | Severe impotence with some function |
| 6 | Mild to moderate impotence |
| 8 | Not impotent but has some loss of function |
| 10 | Not impotent with full function |

TABLE 7
Patient Enrollment by Impotence Classification

| | Severe | Mild to Moderate | Not Impotent | Total |
|---|---|---|---|---|
| Double-Blind | 39 | 25 | 0 | 64 |
| Open Label | 63 | 16 | 0 | 79 |
| Total Patients | 102 | 41 | 0 | 143 |

The topical prostaglandin E₁ composition was found to be safe and effective in impotent men with the moderate to severe impotence. The efficacy rate was 64.7% (66/102 patients) in severely impotent men and 100% (41/41 patients) in mild to moderately impotent men. The overall clinical efficacy rate for the study is 74.8% (107/143 patients) as shown in Table 8, below.

TABLE 8
Overall Clinical Efficacy Rates

| | | | Combined |
|---|---|---|---|
| | Double-Blind Portion | Open-Label Portion | Overall Rate |
| Placebo | 4.7% (3/64) | — | 4.7% (3/64) |
| Active | 87.5% (56/64) | 64.6% (51/79) | 74.8% (107/143) |
| | P < 0.001 | | P < 0.001 |

The prostaglandin E₁ topical composition was extremely effective (100%) in the mild to moderate impotent patient population. The mild to moderate impotence class is the most prevalent class and is estimated to represent 70% of all erectile dysfunction complaints. The product was also very effective (64.7%) in the severely impotent study population.

A placebo efficacy response was seen in only 3 of 64 (4.7%) patients studied in the double-blind portion of the study. This is far below the expected rate of approximately 10% as reported in other clinical studies. This low rate is perhaps due to the fact that the majority (63%) of the patients enrolled in the double-blind portion of the study were classified with severe impotence. While 17 of 64 (26.6%) patients showed improvement with the placebo, only three (3) of those patients had sufficient improvement to be assessed as efficacious (8 or 10 on the classification scale).

TABLE 9
Clinical Efficacy Rates by Impotence Classification

| | | Study Sites | | | |
|---|---|---|---|---|---|
| | | | | | Combined |
| | Portion | No. 1 | No. 2 | No. 3 | Efficacy |
| Severely | Double-Blind | 85.7% | 63.6% | No Patients | 79.5% |
| Impotent | | (24/28) | (7/11) | Entered | (31/39) |
| | Open Label | 72.2% | 33.3% | 51.3% | 55.6% |
| | | (13/18) | (2/6) | (20/39) | (35/63) |
| Mild to | Double-Blind | 100% | 100% | No Patients | 100% |
| Moderate | | (2/2) | (23/23) | Entered | (25/25) |
| Impotence | Open Label | 100% | 100% | No Patients | 100% |
| | | ((14/14) | (2/2) | Entered | (16/16) |

The open label efficacy rate was lower than the double-blind efficacy rate (Table 9). This was primarily due to the enrollment of a relatively high number of severely impotent men in the open-label portion of the study as compared to the double-blind portion. (Table 8) Of the men enrolled in the open label portion of the study, 79.7% (63/79) were assessed as severely impotent while only 60.9% (39/64) were assessed as severely impotent on entering the double-blind portion. The efficacy rate among the severely impotent population is expected to be lower because by definition these men have little or no function. Practically, it is expected to be more difficult to move the impotence classification from 0, 2 or 4 up to 8 or 10. While most of the severely impotent men showed significant improvement, 36 men (36/102 or 35.3%) did not have sufficient improvement to be classified as efficacious.

Adverse events observed in this study were mild transient burning or tingling at the application site. No systemic toxic side effects were observed. Also, none of the spouses involved in the studies reported adverse events. None of the patients dropped out of the study or were lost to follow-up

### EXAMPLE 10

### Multiple Use Open Label Clinical Trial

The safety and efficacy of a 0.4% prostaglandin E₁ topical composition (composition D of Example 4 and Table 1, above) was evaluated in an additional study of a total of 56 men at three study sites. Fifty-six (56) male patients with organic erectile dysfunction entered and completed the study. Patients were classified into groups based on their responses to the International Index of Erectile Dysfunction (IIEF) and the pre dose Sexual Encounter Profile (SEP). Forty-nine (49) patients were classified as having mild to moderate erectile dysfunction and 7 patients were classified as having severe erectile dysfunction. Each patient was asked to use from 3 to 10 doses of medication over a four week period in a multiple use, in-home study. The overall efficacy rate for the mild to moderate group was 75%. The results of this study were consistent with the combined overall efficacy rate reported above in Example 9. None of the patients dropped out of this multiple use study and no severe adverse events were noted.

Inclusion Criteria

1 Males, ages 21-70 years, inclusive.

2. Documented history of erectile dysfunction, which is defined as the inability to achieve and maintain an erection of sufficient rigidity for sexual intercourse due to psychogenic, neurogenic or vasculogenic, causes during the previous 6 months. This includes patients who may still have some erections sufficient for intercourse but not consistently, which is the typical complaint of the age onset, mild to moderate impotent man. The diagnosis of erectile dysfunction based on medical history and physical examination.

Exclusion Criteria

1. History of urethral stricture or obstruction.

2. Any combination of findings from history, physical examination or screening studies which indicate pre-existing impairment of heart, liver and/or kidney function (such as congestive heart failure, unstable angina or recent acute myocardial infarction, uncontrolled diabetes, for erectile dysfunction of hormonal origin) which in the investigator's opinion could influence the outcome of the study.

3. History of penile surgery, including penile implant, prostatectomy or cancer of the prostate, penile trauma including paraplegia or quadriplegia.

4. Any condition which might predispose towards priapism, such as sickle cell anemia, multiple myeloma, or leukemia.

5. Hypertension, (sitting diastolic pressure >90 or systolic >150) requiring treatment with other than angiotensin converting enzyme inhibitors (ACE inhibitors).

6. Presence of a sexually transmitted disease as determined by physical examination.

7. Use of a cavernosal injection or external erectile device within 4 weeks prior to entering into this study.

8. Peyronie's Disease or any palpable fibrous scar or plaque on the penis, evidence of curvature during tumescence and rigidity stimulation or an anomaly of the penis skin or mucosa of the glans.

9. Any concomitant medication which are known to interfere with sexual activity such as antidepressants, some antihypertensives, sedatives hormones and some allergy medications.

10. Received any investigational treatment within 30 days of entering into this study.

11. Inability or unwillingness to give informed consent.

The patient population in this study consisted of men in the age range of 49-70 years old.

TABLE 10
Patient Enrollment by Study Sites

| Patients Enrolled On Study Sites | | | |
|---|---|---|---|
| No. 1 | No. 2 | No. 3 | Total |
| 22 | 13 | 21 | 56 |

Clinical efficacy was evaluated from patient history and patient evaluation questionnaires both before and after medication using the International Index of Erectile Function (Table 11) and the Sexual Encounter Profile (SEP) six-point classification scale (Table 12). Each patient was given 10 active doses and asked to take the medication home and attempt intercourse as many times as possible over a 4 week period. The medication was packaged in a specially designed single dose dispenser.

TABLE 11
International Index of Erectile Function

| Classification | Definition |
|---|---|
| <12 | Severe impotence with no function |
| 12-18 | Mild Impotence with very little function |
| 18-24 | Moderate Impotence with some function |
| 24+ | No dysfunction |

The efficacy response rate was determined as the number of intercourse successes out of the total number of intercourse attempts. To be considered a success, a SEP score of 8 to 10 must be achieved after administration of the dose or the patient must have had satisfactory sexual intercourse. Statistical analysis compared before and after response scores using Chi Square statistics. A statistically significant difference (P<0.001) between before and after dosing scores was found for each group of patients receiving active medication.

TABLE 12
Sexual Encounter Profile(SEP): Six-Point Classification Scale for
Assessing the Severity of Male Erectile Dysfunction (Impotence)

| Classification | Definition |
|---|---|
| 0 | Severe impotence with no function |
| 2 | Moderate Impotence with very little function |
| 4 | Moderate Impotence with some function |
| 6 | Mild Impotence |
| 8 | Not impotent but has some loss of function |
| 10 | Not impotent with full function |

The efficacy response rate was determined as the number of intercourse successes out of the total number of intercourse attempts. To be considered a success, a SEP score of 8 to 10 must achieved after administration of the dose or the patient must have had satisfactory sexual intercourse. Statistical analysis compared before and after response scores using Chi Square statistics. A statistically significant difference (P<0.001) between before and after dosing scores was found.

TABLE 13
Patient Enrollment by Impotence Classification

| | Severe | Mild to Moderate | Total |
|---|---|---|---|
| Patients | 7 | 49 | 56 |

TABLE 14
Efficacy per Patient Group

| | Efficacy by Patients | | Efficacy by Attempts | |
|---|---|---|---|---|
| Mild to Moderate | 36/49 | (74%) | 178/239 | (75%) |
| Severe | 4/7 | (57%) | 16/36 | (44%) |

As previously discussed, the prostaglandin E₁ topical composition was extremely effective (75%) in the mild to moderate impotent patient population. The mild to moderate impotence class is the most prevalent class and is estimated to represent 70% of all erectile dysfunction complaints. The product was less effective (44%) in the severely impotent study population; however, a statistically significant difference was noted between the before and after treatment scores in this group. Even though all of the men in the severe group were totally without any erectile function before the study, 4 of the 7 men (57%) had successful intercourse from at least 3 out of the 10 doses.

Adverse events observed in this study were mild transient burning or tingling at the application site. No systemic toxic side effects were observed. Also, none of the spouses involved in the studies reported adverse events. None of the patients dropped out of the study or were lost to follow-up.

The results of this clinical study indicate the use of the prostaglandin E₁ 0.4% topical composition of the present invention for the treatment of mild, moderate to severe impotence is safe and efficacious.

### EXAMPLE 11

### Phase 3 Clinical Studies

Phase 3 Clinical Studies were performed using the topical composition I of Table 1 modified to produce four test compositions having PGE₁ doses of 0 micrograms (mcg) (placebo), 100 mcg, 200 mcg or 300 mcg.

The enrolled patients received a four week non-treatment period, then were randomly assigned one of four treatment groups: placebo, 100 mcg, 200 mcg or 300 mcg for the 12 week take-home portion of the study.

The inclusion criteria were: the patients were at least 21 years of age with no upper age limit, a history of erectile dysfunction three months or longer in duration, an IIEF erectile function domain score less than or equal to 25, and at least 4 attempts of sexual intercourse during the non-treatment period.

Any patient with an erectile dysfunction caused by untreated endocrine disease, significant penile pathology, a history of orthostatic hypotension, syncopal episodes, or pre-syncopal symptoms (previous 6 months), clinically significant hepatic or renal disease, a history of myocardial infarction (within the previous 6 months), a significant neurological disease such as stroke or spinal cord injury, currently taking prescription or over the counter erectile dysfunction medication or therapy, or have a history of allergy to alprostadil was excluded from the study.

Patients who were not excluded from the studies were patients with a history of prostatectomy, patients with controlled diabetes, patients taking nitrate medications and alpha blockers, and patients with a history of efficacy failure with oral PDE5 inhibitor therapy (sildenafil, Viagra™). The demographic and baseline characteristics of the Phase 3 treatment group are summarized in Table 15 below. A given patient may have had more than one co-morbid condition; the sum of the percents under “Medical History” is more than 100%.

Phase 3 clinical studies were based on a study design of randomized, placebo controlled, double-blind, and parallel treatment design involving a take-home study in mild to severe erectile dysfunction patient. There were two separate studies performed in the United States at 85 sites. The patient population consisted of 1732 men 24-87 years of age. Of the starting population, 1410 completed the study, a 18% drop out rate. The patients were randomly assigned to four parallel treatment groups: placebo, 100 microgram, 200 microgram, and 300 microgram prostaglandin E₁ dose. An initial 4 week non-treatment period was followed by 12 weeks of take-home dosing. Up to 25 doses per patient were self-administered over the twelve week treatment period. The patients in the study were mild to severe erectile dysfunction patients, having IIEF erectile dysfunction domain scores less than or equal to 25. Questions 1, 2, 3, 4, 5, and 15 of the IIEF were entered into the study.

The demographics of the patient population are summarized in Table 15, below. Note that a given patient may have more than one co-morbid condition in his medical history.

TABLE 15
Treatment Group Demographics
Mean Age: 59.6 years (1732 men, 23-87 yrs. of age)

| Age > 65 | 15% |
|---|---|
| Baseline Erectile Function Domain Score | 13.8 |
| History of ED > 1 yr | 93% |
| Medical History | |
| Diabetes | 21% |
| Hypertension | 44% |
| Cardiac Disease | 28% |
| Nitrates and alpha blockers | 16% |
| Prostatectomy | 12% |
| Oral Sildenafil Efficacy Failures | 18% |

The composition of the four treatment groups is summarized in Table 16, below. As noted above, a patient may have had more than one co-morbid condition.

TABLE 16
Demographic and Baseline Characteristics

| | | Treatment Group | | | |
|---|---|---|---|---|---|
| | Placebo | 100 mcg | 200 mcg | 300 mcg | All PGE₁ |
| | (N = 434) | (N = 434) | (N = 430) | (N = 434) | (N = 1732) |
| | Mean age, year | | | | |
| | (range) | | | | |
| | 61 (31-87) | 61 (35-86) | 60 (24-87) | 61 (23-86) | 61 (31-87) |
| Medical History | | | | | |
| Hypertension | 191 | 186 | 180 | 198 | 755 |
| Coronary | 118 | 107 | 125 | 135 | 485 |
| artery disease | | | | | |
| Diabetes | 85 | 90 | 95 | 86 | 356 |
| mellitus | | | | | |
| Prostatectomy | 46 | 71 | 45 | 51 | 213 |
| Sildenafil | 80 | 83 | 71 | 78 | 312 |
| (Low or no | | | | | |
| efficacy) | | | | | |

The measures used a primary efficacy endpoints were: the change in the International Index of Erectile Function (IIEF) Erectile Function Domain score from baseline to final visit compared to placebo; Question 2 of the Sexual Encounter Profile (SEP): “Were you able to insert your penis into your partner's vagina?”; and Question 3 of the SEP: “Did your erection last long enough for you to complete intercourse with ejaculation?” For the response to Q2 and Q3, mean per patient scores were compared to placebo.

Secondary efficacy end points included a Global Assessment Question (GAQ): “Did your erections improve while on the study medication?” as well as the scores on other domains on the IIEF. Erectile dysfunction severity was characterized by IIEF erectile function domain scores as follows: severe (<11), moderate (11-16), mild to moderate (17-21), mild (22-25) or normal (>26).

The efficacy of the four treatments as measured by the change in the erectile function domain of the IIEF is shown in Table 17 for the population of patients who completed the study. The two highest dosage levels produced the largest effect. The differences in scores compared to placebo were significant at the 0.001 level for all dosages.

TABLE 17
Erectile Function Domain

| | Mean Change | | |
|---|---|---|---|
| | from | Difference | Number |
| | Baseline | Significance | of |
| | to Endpoint | vs. Placebo | Patients |
| Placebo | −0.7 | | N = 408 |
| 100 mcg | 1.6 | (p < 0.001) | N = 421 |
| 200 mcg | 2.5 | (p < 0.001) | N = 405 |
| 300 mcg | 2.4 | (p < 0.001) | N = 417 |

Similarly, the responses to the global assessment question, “When using the study medication, did you feel your erections improved? (Table 18 below) also showed differences from placebo at p<0.001 level for all dosage levels of PGE₁.

TABLE 18
Global Assessment Question
When using the study medication, did you feel your erections improved?

| | Percent | Difference | Number |
|---|---|---|---|
| | Patient | Significance | of |
| | Improvement | vs. Placebo | Patients |
| Placebo | 20 | | N = 394 |
| 100 mcg | 40 | (p < 0.001) | N = 408 |
| 200 mcg | 47 | (p < 0.001) | N = 392 |
| 300 mcg | 52 | (p < 0.001) | N = 398 |

The responses to question 2 of the Sexual Encounter Profile (SEP) regarding the ability to insert the penis into the partner's vagina, (Table 19 below) also showed differences significant at the p≦0.001 level for all PGE, doses compared to placebo.

TABLE 19
Sexual Encounter Profile (SEP) Scores
(Ability to insert penis in partner's vagina)

| | Percent | | |
|---|---|---|---|
| | Patient | Difference | Number |
| | Intercourse | Significance | of |
| | Success Rate | vs. Placebo | Patients |
| Placebo | 51 | | N = 411 |
| 100 mcg | 57 | (p = 0.001) | N = 418 |
| 200 mcg | 58 | (p < 0.001) | N = 410 |
| 300 mcg | 58 | (p < 0.001) | N = 410 |

The responses to question 3 of the SEP regarding the ability to maintain an erection to ejaculation (Table 20, below) were significantly different from control for the two higher doses (p<0.001), and significant at the 0.003 level for the 100 microgram dose.

TABLE 20
Sexual Encounter Profile (SEP) Scores
(Ability to maintain erection to ejaculation)

| | Percent | | |
|---|---|---|---|
| | Patient | Difference | Number |
| | Intercourse | Significance | of |
| | Success Rate | vs. Placebo | Patients |
| Placebo | 30 | | N = 411 |
| 100 mcg | 39 | (p = 0.003) | N = 418 |
| 200 mcg | 42 | (p < 0.001) | N = 410 |
| 300 mcg | 39 | (p < 0.001) | N = 410 |

A summary of the primary and secondary efficacy endpoints is found in Table 21, below. The overall change with PGE₁ treatment is significantly different from placebo.

TABLE 21
Summary of Major Efficacy Variables at Endpoint

| | | | Treatment Groups | | | | | | |
|---|---|---|---|---|---|---|---|---|---|
| | Placebo | | 100 mcg | | 200 mcg | | 300 mcg | | Overall |
| | (N = 416) | | (N = 422) | | (N = 412) | | (N = 419) | | P |
| | EP | change | EP | change | EP | change | EP | change | Value |
| Primary Efficacy | | | | | | | | | |
| Endpoints | | | | | | | | | |
| IIEF, erectile function, | 13.3 | −0.7 | 15.3 | 1.6 | 16.1 | 2.5 | 16.1 | 2.5 | <0.001 |
| mean change from | | | | | | | | | |
| baseline | | | | | | | | | |
| SEP Scores, mean % | | | | | | | | | |
| success rate per patient | | | | | | | | | |
| from baseline | | | | | | | | | |
| Question 2 | 51.2 | −4.5 | 56.6 | 2.9 | 58.2 | 5.1 | 57.5 | 7.2 | 0.002 |
| (Ability to achieve | | | | | | | | | |
| erections) | | | | | | | | | |
| Question 3 | 30.2 | 0.4 | 38.9 | 7.0 | 41.9 | 13.8 | 38.5 | 9.1 | <0.001 |
| (Ability to maintain | | | | | | | | | |
| erections to ejaculation) | | | | | | | | | |
| Secondary Efficacy | | | | | | | | | |
| Endpoints | | | | | | | | | |
| Global Assessment | 20 | | 40 | | 47 | | 52 | | <0.001 |
| Question, % of patients | | | | | | | | | |
| answering “yes” (Did the | | | | | | | | | |
| medication improve your | | | | | | | | | |
| erections? Y/N) | | | | | | | | | |

The percent of patients achieving a normal IIEF score (>26) after treatment was 6% (placebo), 10% (100 mcg), 14% (200 mcg) and 16% (300 mcg).

### EXAMPLE 12

### Treatment of Erectile Dysfunction in Diabetic Patients

The measures of efficacy of the treatment in the subset of patients who had a medical history of diabetes mellitus are summarized in Table 22-23, below. The efficacy of the four treatments as measured by the change in the erectile function domain of the IIEF is shown in Table 22 for the subset of diabetes patients who completed the study. The two highest dosage levels produced the largest effect. The differences in scores compared to placebo were significant at the p<0.05 level for all dosages.

TABLE 22
Diabetes Patients
Erectile Function Domain

| | Mean Change | | |
|---|---|---|---|
| | from | Difference | Number |
| | Baseline | Significance | of |
| | to Endpoint | vs. Placebo | Patients |
| Placebo | −1.2 | | N = 81 |
| 100 mcg | 2.1 | (p = 0.014) | N = 90 |
| 200 mcg | 3.7 | (p < 0.001) | N = 94 |
| 300 mcg | 2.4 | (p = 0.003) | N = 95 |

The responses to the global assessment question, “When using the study medication, did you feel your erections improved? (Table 23 below) showed differences from placebo at the p<0.001 level for all dosage levels of PGE₁.

TABLE 23
Diabetes Patients
Global Assessment Question
When using the study medication, did you feel your erections improved?

| | Percent | Difference | Number |
|---|---|---|---|
| | Patient | Significance | of |
| | Improvement | vs. Placebo | Patients |
| Placebo | 20 | | N = 76 |
| 100 mcg | 43 | (p = 0.001) | N = 69 |
| 200 mcg | 45 | (p = 0.001) | N = 92 |
| 300 mcg | 53 | (p < 0.001) | N = 79 |

The responses to question 2 of the Sexual Encounter Profile (SEP) regarding the ability to insert the penis into the partner's vagina, (Table 24 below) did not show differences significant at the p≦0.05 level for any PGE₁ doses compared to placebo.

TABLE 24
Diabetes Patients
Sexual Encounter Profile (SEP) Scores
(Ability to insert penis in partner's vagina)

| | Percent | | |
|---|---|---|---|
| | Patient | Difference | Number |
| | Intercourse | Significance | of |
| | Success Rate | vs. Placebo | Patients |
| Placebo | 44 | | N = 84 |
| 100 mcg | 51 | p = 0.209 | N = 90 |
| 200 mcg | 64 | p = 0.069 | N = 95 |
| 300 mcg | 52 | p = 0.255 | N = 83 |

The responses to question 3 of the SEP regarding the ability to maintain an erection to ejaculation (Table 25, below) were significantly different from control for the 200 mcg dose (p<0.005).

TABLE 25
Diabetes Patients
Sexual Encounter Profile (SEP) Scores
(Ability to maintain erection to ejaculation)

| | Percent | | |
|---|---|---|---|
| | Patient | Difference | Number |
| | Intercourse | Significance | of |
| | Success Rate | vs. Placebo | Patients |
| Placebo | 29 | | N = 84 |
| 100 mcg | 36 | p = 0.123 | N = 90 |
| 200 mcg | 46 | p = 0.002 | N = 95 |
| 300 mcg | 33 | p = 0.253 | N = 83 |

### EXAMPLE 13

### Treatment of Erectile Dysfunction in Prostatectomy Patients

The measures of efficacy of the treatment of the subset of patients who had a medical history of prostatectomy are summarized in Tables 26-29, below. The efficacy of the four treatments as measured by the change in the erectile function domain of the IIEF is shown in Table 27 for this subset of patients who completed the study. The two highest dosage levels produced the largest effect. The differences in scores compared to placebo were significant at the p<0.01 level for all dosages.

TABLE 26
Prostatectomy Patients
Erectile Function Domain

| | Mean | | |
|---|---|---|---|
| | Change | | |
| | from | Difference | |
| | Baseline to | Significance | Number of |
| | Endpoint | vs. Placebo | Patients |
| Placebo | −2.2 | | N = 46 |
| 100 mcg | 2.2 | p = 0.004 | N = 71 |
| 200 mcg | 2.4 | p = 0.006 | N = 44 |
| 300 mcg | 2.5 | p = 0.003 | N = 51 |

The responses to the global assessment question, “When using the study medication, did you feel your erections improved? (Table 27 below) showed differences from placebo at the p<0.001 level for all dosage levels of PGE₁.

TABLE 27
Prostatectomy Patients
Global Assessment Question
When using the study medication, did you feel your erections
improved?

| | Percent | Difference | |
|---|---|---|---|
| | Patient | Significance | Number of |
| | Improvement | vs. Placebo | Patients |
| Placebo | 11 | | N = 44 |
| 100 mcg | 47 | p < 0.001 | N = 70 |
| 200 mcg | 57 | p < 0.001 | N = 42 |
| 300 mcg | 55 | p < 0.001 | N = 51 |

The responses to question 2 of the Sexual Encounter Profile (SEP) regarding the ability to insert the penis into the partner's vagina, (Table 28 below) only showed differences significant at the p<0.01 level for 300 mcg PGE₁ dose compared to placebo.

TABLE 28
Prostatectomy Patients
Sexual Encounter Profile (SEP) Scores
(Ability to insert penis in partner's vagina)

| | Percent Patient | Difference | |
|---|---|---|---|
| | Intercourse | Significance | Number of |
| | Success Rate | vs. Placebo | Patients |
| Placebo | 21 | | N = 45 |
| 100 mcg | 41 | p = 0.129 | N = 71 |
| 200 mcg | 35 | p = 0.155 | N = 45 |
| 300 mcg | 36 | p = 0.006 | N = 51 |

The responses to question 3 of the SEP regarding the ability to maintain an erection to ejaculation (Table 29, below) were significantly different from control at the p=0.056 level for the 300 mcg dose.

TABLE 29
Prostatectomy Patients
Sexual Encounter Profile (SEP) Scores
(Ability to maintain erection to ejaculation)

| | Percent Patient | Difference | |
|---|---|---|---|
| | Intercourse | Significance | Number of |
| | Success Rate | vs. Placebo | Patients |
| Placebo | 13 | | N = 45 |
| 100 mcg | 22 | p = 0.541 | N = 71 |
| 200 mcg | 24 | p = 0.511 | N = 45 |
| 300 mcg | 24 | p = 0.056 | N = 51 |

### EXAMPLE 14

### Regimen of Treatment of Erectile Dysfunction in Prostatectomy Patients

Patients needing treatment for erectile dysfunction following radical prostatectomy or nerve-sparing prostatectomy are treated with the semisolid prostaglandin composition of Example 11. While treatment can be started at any time after prostatectomy surgery, it is preferred that treatment start about one to about six months after surgery. Depending on the severity of erectile dysfunction as assessed using the IIEF, a regular regimen of treatment not necessarily linked to anticipated sessions of sexual intercourse is administered. A semi-solid vasoactive prostaglandin composition delivering a dose of about 100 mcg to about 400 mcg prostaglandin E₁ is applied to the fossa navicularis of the penis at least twice a week, every other day, or on a daily basis. For “on demand” treatment, the semi-solid vasoactive prostaglandin composition is applied to the fossa navicularis of the penis about 2-30 minutes before sexual intercourse, preferably about 5-15 minutes before sexual intercourse. About 40 to about 60 percent of the treated patients report that they feel that their erections have improved while taking the medication after about three months of treatment.

### EXAMPLE 15

### Treatment of Erectile Dysfunction in Erectile Dysfunction Patients for Whom Oral Phosphodiesterase-5 Inhibitor Therapy Was Ineffective

The measures of efficacy of the treatment in the treatment in the subset of erectile dysfunction patients for whom oral phosphodiesterase-5 inhibitor therapy was ineffective are summarized in Tables 30-33, below. The efficacy of the four treatments as measured by the change in the erectile function domain of the IIEF is shown in Table 31 for this subset of patients who completed the study. The two highest dosage levels produced the largest effect. The differences in scores compared to placebo were not significant at the p<0.05 level for all dosages.

TABLE 30
Erectile Dysfunction Patients For Whom Oral Sildenafil
Treatment was Ineffective
Erectile Function Domain

| | | Difference | |
|---|---|---|---|
| | Mean Change from | Significance vs. | Number of |
| | Baseline to Endpoint | Placebo | Patients |
| Placebo | −0.4 | | N = 80 |
| 100 mcg | 1.2 | p = 0.134 | N = 83 |
| 200 mcg | 1.7 | p < 0.061 | N = 70 |
| 300 mcg | 1.4 | p < 0.097 | N = 78 |

The responses to the global assessment question, “When using the study medication, did you feel your erections improved? (Table 31 below) showed differences from placebo at the p<0.05 level for the 200 mcg and 300 mcg dosage levels of PGEI.

TABLE 31
Erectile Dysfunction Patients For Whom Oral Sildenafil
Treatment was Ineffective
Global Assessment Question
When using the study medication, did you feel
your erections improved?

| | Percent | Difference | |
|---|---|---|---|
| | Patient | Significance | Number of |
| | Improvement | vs. Placebo | Patients |
| Placebo | 21 | | N = 77 |
| 100 mcg | 29 | p = 0.158 | N = 82 |
| 200 mcg | 37 | p = 0.025 | N = 68 |
| 300 mcg | 45 | p = 0.001 | N = 73 |

The responses to question 2 of the Sexual Encounter Profile (SEP) regarding the ability to insert the penis into the partner's vagina, (Table 32 below) showed differences significant at the p≦0.05 level for the 200 mcg and 300 mcg PGE₁ doses compared to placebo.

TABLE 32
Erectile Dysfunction Patients For Whom Oral Sildenafil
Treatment was Ineffective
Sexual Encounter Profile (SEP) Scores
(Ability to insert penis in partner's vagina)

| | Percent Patient | Difference | |
|---|---|---|---|
| | Intercourse | Significance | Number of |
| | Success Rate | vs. Placebo | Patients |
| Placebo | 43 | | N = 80 |
| 100 mcg | 45 | p = 0.181 | N = 83 |
| 200 mcg | 45 | p = 0.046 | N = 70 |
| 300 mcg | 49 | p = 0.004 | N = 78 |

The responses to question 3 of the SEP regarding the ability to maintain an erection to ejaculation (Table 33, below) were not significantly different from control.

TABLE 33
Erectile Dysfunction Patients For Whom Oral Sildenafil
Treatment was Ineffective
Sexual Encounter Profile (SEP) Scores
(Ability to maintain erection to ejaculation)

| | Percent Patient | Difference | |
|---|---|---|---|
| | Intercourse Success | Significance vs. | Number of |
| | Rate | Placebo | Patients |
| Placebo | 23 | | N = 80 |
| 100 mcg | 29 | p = 0.809 | N = 83 |
| 200 mcg | 32 | p = 0.112 | N = 70 |
| 300 mcg | 30 | p = 0.820 | N = 78 |

### EXAMPLE 16

### Treatment of Erectile Dysfunction in Hypertensive Patients

The measures of efficacy of the treatment in the subset of patients who had a medical history of hypertension are summarized in Tables 34-37, below. The efficacy of the four treatments as measured by the change in the erectile function domain of the IIEF is shown in Table 35 for this subset of patients who completed the study. The two highest dosage levels produced the largest effect. The differences in scores compared to placebo were significant at the p<0.05 level for all dosages, and significant at the p<0.001 level for the 200 mcg and 300 mcg doses.

TABLE 34
Erectile Function Domain

| | Mean Change from | Difference | |
|---|---|---|---|
| | Baseline to | Significance | Number of |
| | Endpoint | vs. Placebo | Patients |
| Placebo | −0.6 | | N = 187 |
| 100 mcg | 1 | p = 0.022 | N = 185 |
| 200 mcg | 2.9 | p < 0.001 | N = 175 |
| 300 mcg | 2.0 | p < 0.001 | N = 198 |

The responses to the global assessment question, “When using the study medication, did you feel your erections improved? (Table 35 below) showed differences from placebo at the p<0.001 level for all dosage levels of PGEI.

TABLE 35
Global Assessment Question
When using the study medication, did you
feel your erections improved?

| | Percent Patient | Difference Significance | Number of |
|---|---|---|---|
| | Improvement | vs. Placebo | Patients |
| Placebo | 20 | | N = 178 |
| 100 mcg | 37 | p < 0.001 | N = 178 |
| 200 mcg | 47 | p < 0.001 | N = 173 |
| 300 mcg | 49 | p < 0.001 | N = 187 |

The responses to question 2 of the Sexual Encounter Profile (SEP) regarding the ability to insert the penis into the partner's vagina, (Table 36 below) also showed differences significant at the p≦0.05 level compared to placebo for all PGE, doses.

TABLE 36
Sexual Encounter Profile (SEP) Scores
(Ability to insert penis in partner's vagina)

| | Percent Patient Intercourse | Difference Sign. vs. | |
|---|---|---|---|
| | Success Rate | Placebo | Number |
| Placebo | 47 | | N = 189 |
| 100 mcg | 53 | p = 0.038 | N = 182 |
| 200 mcg | 59 | p < 0.001 | N = 179 |
| 300 mcg | 54 | p = 0.006 | N = 195 |

The responses to question 3 of the SEP regarding the ability to maintain an erection to ejaculation (Table 37, below) were significantly different from control only for the 200 mcg dose (p=0.001).

TABLE 37
Sexual Encounter Profile (SEP) Scores
(Ability to maintain erection to ejaculation)

| | Percent Patient | Difference | |
|---|---|---|---|
| | Intercourse Success | Significance vs. | Number of |
| | Rate | Placebo | Patients |
| Placebo | 26 | | N = 189 |
| 100 mcg | 35 | p = 0.329 | N = 182 |
| 200 mcg | 41 | p = 0.001 | N = 179 |
| 300 mcg | 35 | p = 0.34 | N = 195 |

### EXAMPLE 17

### Treatment of Erectile Dysfunction in Cardiac Patients

The measures of efficacy of the treatment in the subset of patients who had a medical history of cardiac disease are summarized in Tables 38-41, below. The efficacy of the four treatments as measured by the change in the erectile function domain of the IIEF is shown in Table 38 for this subset of patients who completed the study. The two highest dosage levels produced the largest effect. The differences in scores compared to placebo were significant at the p<0.005 level for all dosages.

TABLE 38
Erectile Function Domain

| | Mean Change from | Difference | |
|---|---|---|---|
| | Baseline to | Significance vs. | Number of |
| | Endpoint | Placebo | Patients |
| Placebo | −1.7 | | N = 115 |
| 100 mcg | 1.4 | p = 0.002 | N = 107 |
| 200 mcg | 1.5 | p = 0.001 | N = 122 |
| 300 mcg | 1.9 | p < 0.001 | N = 135 |

The responses to the global assessment question, “When using the study medication, did you feel your erections improved? (Table 39 below) showed differences from placebo at the p<0.005 level for all dosage levels of PGE₁.

TABLE 39
Global Assessment Question
When using the study medication, did you feel your erections
improved?

| | Percent | Difference | |
|---|---|---|---|
| | Patient | Significance | Number of |
| | Improvement | vs. Placebo | Patients |
| Placebo | 17 | | N = 111 |
| 100 mcg | 35 | p < 0.003 | N = 104 |
| 200 mcg | 48 | p < 0.001 | N = 116 |
| 300 mcg | 50 | p < 0.001 | N = 126 |

The responses to question 2 of the Sexual Encounter Profile (SEP) regarding the ability to insert the penis into the partner's vagina, (Table 40 below) also showed differences significant at the p<0.05 level compared to placebo only at the 200 mcg dose, although the difference compared to control was significant at the p=0.054 level for the 300 mcg PGE₁.

TABLE 40
Sexual Encounter Profile (SEP) Scores
(Ability to insert penis in partner's vagina)

| | Percent Patient | Difference | |
|---|---|---|---|
| | Intercourse | Significance | Number of |
| | Success Rate | vs. Placebo | Patients |
| Placebo | 44 | | N = 117 |
| 100 mcg | 54 | p = 0.109 | N = 105 |
| 200 mcg | 54 | p = 0.018 | N = 125 |
| 300 mcg | 51 | p = 0.054 | N = 130 |

The responses to question 3 of the SEP regarding the ability to maintain an erection to ejaculation (Table 41, below) were significantly different from control for the two higher doses (p<0.05).

TABLE 41
Sexual Encounter Profile (SEP) Scores
(Ability to maintain erection to ejaculation)

| | Percent Patient | Difference | |
|---|---|---|---|
| | Intercourse Success | Significance vs. | Number of |
| | Rate | Placebo | Patients |
| Placebo | 23 | | N = 117 |
| 100 mcg | 33 | p = 0.260 | N = 105 |
| 200 mcg | 36 | p = 0.003 | N = 125 |
| 300 mcg | 31 | p = 0.022 | N = 130 |

### EXAMPLE 18

### Efficacy of Treatment Measures by Baseline Severity of Erectile Dysfunction

The efficacy of treatment compared to the initial baseline severity was analyzed separately for the two studies comprising the Phase 3 study. Baseline severity was defined by IIEF Erectile Function domain scores as follows: severe (1-10), Moderate (11-18), Mild to Moderate (17-21), Mild (23-25). The results are summarized in Tables 42-46, below.

TABLE 42
Change from Baseline in IIEF EF Domain Scores at Endpoint by
Baseline Severity (Study 1)

| | | Mild to | | |
|---|---|---|---|---|
| | Mild | Moderate | Moderate | Severe |
| Placebo | −4 | −1.9 | −0.4 | 2.5 |
| 100 mcg | −0.5 | −0.7 | 2.3 | 4.1 |
| 200 mcg | 1 | 1.1 | 2.9 | 4.9 |
| 300 mcg | −0.2 | 1.6 | 3.8 | 5.1 |

TABLE 43
Change from Baseline in IIEF EF Domain Scores at Endpoint by
Baseline Severity (Study 2)

| | | Mud to | | |
|---|---|---|---|---|
| | Mild | Moderate | Moderate | Severe |
| Placebo | −4.8 | −0.7 | −0.1 | 0.6 |
| 100 mcg | −1.6 | 0.3 | 1.5 | 4.6 |
| 200 mcg | −3.1 | 2.9 | 2.7 | 2.5 |
| 300 mcg | −0.4 | 1.2 | 1.5 | 3.4 |

TABLE 44
Treatment on Vaginal Penetration Success
Rates Baseline ED ED Severity
SEPQ2/SEPQ1 (Percent Penetration Successes/Attempts) (Study 2)

| | | Mild to | | |
|---|---|---|---|---|
| | Mild | Moderate | Moderate | Severe |
| Placebo | 77.7 | 68.3 | 51.2 | 23.1 |
| 100 mcg | 90.5 | 72.8 | 60.4 | 25.9 |
| 200 mcg | 83.9 | 82.5 | 62.6 | 25.4 |
| 300 mcg | 88.4 | 78.9 | 63.8 | 33.2 |

TABLE 45
Treatment on Successful Intercourse Completion Rates by Baseline
ED Severity
SEPQ3/SEPQ1 (Number of completions to ejaculation/Attempts)
Percent of Patients with Success (Study 2)

| | | Mild to | | |
|---|---|---|---|---|
| | Mild | Moderate | Moderate | Severe |
| Placebo | 62.6 | 47.6 | 27.4 | 10.3 |
| 100 mcg | 73.5 | 48.6 | 38.2 | 17.3 |
| 200 mcg | 71.9 | 63.7 | 41.4 | 23.9 |
| 300 mcg | 75.7 | 61.9 | 37.7 | 15.4 |

### EXAMPLE 19

### Integrated Safety Analysis

The results of the integrated safety analysis are presented in Tables 46-47, below.

TABLE 46
Summary of Treatment Related Adverse Events

| | Treatment Group | | | | |
|---|---|---|---|---|---|
| | Placebo | 100 mcg | 200 mcg | 300 mcg | Overall |
| | N = 434 | N = 434 | N = 434 | N = 434 | N = 1732 |
| Patients with a | 61 (14) | 156 (36) | 187 (20) | 188 (42) | 592 (34) |
| least one drug | | | | | |
| related adverse | | | | | |
| event, n (%) | | | | | |
| Drug related | 0 | 0 | 0 | 0 | 0 |
| serious adverse | | | | | |
| events | | | | | |
| Patients | 1 (1) | 10 (2) | 14 (3) | 26 (6) | 51 (3) |
| withdrawn | | | | | |
| due to drug | | | | | |
| related adverse | | | | | |
| events, n (%) | | | | | |
| Drug related | | | | | |
| adverse events | | | | | |
| occurring | | | | | |
| in ≧3% of | | | | | |
| all patients | | | | | |
| Penile Burning | 26 (6) | 76 (18) | 106 (25) | 101 (23) | 309 (18) |
| Penile | 9 (2) | 33 (8) | 39 (9) | 49 (11) | 121 (7) |
| Erythema | | | | | |
| Genital Pain | 2 (0.5) | 48 (11) | 67 (16) | 76 (18) | 193 (11) |
| Vaginal | 7 (2) | 15 (3) | 30 (7) | 18 (4) | 70 (4) |
| Burning | | | | | |

TABLE 47
Summary of Patients Discontinued Due to Drug Related
Adverse Events

| | Number of Patients |
|---|---|
| Adverse Event | Discontinued (%) |
| Genital Pain | 15 (0.9) |
| Penile Burning | 21 (1.2) |
| Penile Erythema | 4 (0.2) |
| Vaginal Burning | 3 (0.3) |
| Other | 8 (0.5) |
| Total, n(%) | 51 (3) |

In general, the treatment produced no serious side effects. Most adverse events were localized to the site of application but were mild, short in duration and well tolerated. Overall, the treatment demonstrated efficacy across a broad range of erectile dysfunction severity and co-morbid conditions.

### EXAMPLE 20

### Treatment of Hypertension Patients Also Receiving Alpha₁ Blockers

The measures of efficacy of the treatment in the subset of patients who had a medical history of hypertension and who were also being treated with alpha₁ blockers are summarized in Tables 48-51, below. The small sample size limits the calculation of the p values for comparison of differences.

The efficacy of the four treatments as measured by the change in the erectile function domain of the IIEF is shown in Table 48 for this subset of patients who completed the study. The 300 mcg dosage level produced the largest effect. The differences in scores compared to placebo were not significant at the p<0.05 level for any dosages.

TABLE 48
Erectile Function Domain

| | Mean Change from | Difference Significance | Number of |
|---|---|---|---|
| | Baseline to Endpoint | vs. Placebo | Patients |
| Placebo | −1.2 | | N = 27 |
| 100 mcg | 0.0 | p < 0.619 | N = 24 |
| 200 mcg | −0.2 | p < 0.543 | N = 26 |
| 300 mcg | 2.2 | p < 0.112 | N = 25 |

In spite of the small sample size, the responses to the global assessment question, “When using the study medication, did you feel your erections improved? (Table 49 below) showed differences from placebo at the p<0.05 level for the 300 mcg dosage level.

TABLE 49
Global Assessment Question When using
the study medication, did you feel your erections improved?

| | Percent Patient | Difference Significance vs. | Number of |
|---|---|---|---|
| | Improvement | Placebo | Patients |
| Placebo | 11.5 | | N = 26 |
| 100 mcg | 25 | p < 0.474 | N = 24 |
| 200 mcg | 28 | p < 0.103 | N = 25 |
| 300 mcg | 44 | p < 0.033 | N = 25 |

The responses to question 2 of the Sexual Encounter Profile (SEP) regarding the ability to insert the penis into the partner's vagina, (Table 50 below) did not show differences compared to placebo that were not at the p<0.05 level for any dose levels. The 200 and 300 mcg dose levels produced the largest effects.

TABLE 50
Sexual Encounter Profile (SEP) Scores
(Ability to insert penis in partner's vagina)

| | Percent Patient | Mean Change | Difference | Number |
|---|---|---|---|---|
| | Intercourse Success | Compared to | Significance | of |
| | Rate | Baseline | vs. Placebo | Patients |
| Placebo | 52.8 | −5.6 | | N = 28 |
| 100 mcg | 39.8 | 2.4 | p < 0.266 | N = 24 |
| 200 mcg | 47.3 | 4.8 | p < 0.327 | N = 26 |
| 300 mcg | 59.5 | 6.7 | p < 0.151 | N = 26 |

The responses to question 3 of the SEP regarding the ability to maintain an erection to ejaculation (Table 51, below) were not significantly different from placebo.

TABLE 51
Sexual Encounter Profile (SEP) Scores (Ability to
maintain erection to ejaculation)

| | Percent Patient | Mean Change | Difference | |
|---|---|---|---|---|
| | Intercourse | Compared to | Significance | Number of |
| | Success Rate | Baseline | vs. Placebo | Patients |
| Placebo | 29.0 | 2.8 | | N = 28 |
| 100 mcg | 30.8 | 12.0 | p < 0.140 | N = 24 |
| 200 mcg | 34.3 | 14.0 | p < 0.317 | N = 26 |
| 300 mcg | 40.1 | 1.5 | p < 0.559 | N = 26 |

A similar analysis on a smaller subset of patients who had a medical history of hypertension and who were also being treated with nitrates provided no useful information due to small sample size (a total of 22 patients in all four treatment groups).

The claims should not be read as limited to the described order or elements unless stated to that effect. Therefore, all embodiments that come within the scope and spirit of the following claims and equivalents thereto are claimed as the invention.

We claim:

1. A method of treating erectile dysfunction in a patient suffering from a co-morbid condition comprising the step of:
placing an erection-inducing amount of a semi-solid composition in the fossa navicularis of the patient, the semi-solid composition comprising:
a vasoactive prostaglandin;
a penetration enhancer;
a polymeric thickener selected from the group consisting of a polysaccharide gum and a polyacrylic acid polymer;
a lipophilic component selected from the group consisting of an aliphatic C₁ to C₈ alcohol, an aliphatic C₈ to C₃₀ ester, and a mixture thereof; and
an acidic buffer system.

2. The method in accordance with claim 1 wherein the vasoactive prostaglandin is selected from the group consisting of PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF₃, and mixtures thereof.

3. The method in accordance with claim 1 wherein the vasoactive prostaglandin is prostaglandin E₁.

4. The method in accordance with claim 1 wherein the vasoactive prostaglandin is present in the amount of 0.001 weight percent to about 1 weight percent, based on the total weight of the composition.

5. The method in accordance with claim 1 wherein the vasoactive prostaglandin is present in the amount of about 0.07 weight percent to about 0.4 weight percent, based on the total weight of the composition.

6. The method of claim 1 wherein the semi-solid composition is packaged as a unit dose and the vasoactive prostaglandin is present in the amount of about 0.05 mg to about 0.8 mg per unit dose.

7. The method in accordance with claim 1 wherein the polymeric thickener is a polyacrylic acid polymer.

8. The method in accordance with claim 1 wherein the polymeric thickener is a shear-thinning polysaccharide gum.

9. The method in accordance with claim 8 wherein the shear-thinning polysaccharide gum is a galactomannan gum.

10. The method in accordance with claim 8 wherein the shear-thinning polysaccharide gum is a modified galactomannan gum.

11. The method in accordance with claim 10 wherein the modified galactomannan gum is a modified guar gum.

12. The method in accordance with claim 1 wherein the composition has a viscosity of about 5,000 cps to about 20,000 cps.

13. The method in accordance with claim 1 wherein the composition has a viscosity of about 7,000 cps to about 13,000 cps.

14. The method in accordance with claim 1 wherein the penetration enhancer is selected from the group consisting of an alkyl-2-(N-substituted amino)-alkanoate, an alkyl-2-(N,N-disubstituted amino)-alkanoate, an (N-substituted amino)-alkanol alkanoate, an (N,N-disubstituted amino)-alkanol alkanoate, pharmaceutically acceptable salts thereof and mixtures thereof.

15. The method in accordance with claim 1 wherein the penetration enhancer is dodecyl 2-(N,N-dimethylamino)-propionate hydrochloride.

16. The method in accordance with claim 1 wherein the lipophilic component comprises at least one aliphatic C₈ to C₃₀ ester.

17. The method in accordance with claim 1 wherein the lipophilic component comprises at least one glyceryl ester selected from the group consisting monoglycerides, diglycerides, triglycerides, and mixtures thereof.

18. The method in accordance with claim 1 wherein the lipophilic component comprises at least one glyceryl ester selected from the group consisting of glyceryl monooleate, triolein, trimyristin, tristearin, and mixtures thereof.

19. The method in accordance with claim 1 wherein the acidic buffer system provides a buffered pH value for said composition in the range of about 3 to about 7.4.

20. The method in accordance with claim 1 wherein the acidic buffer system provides a buffered pH value for said composition in the range of about 3 to about 6.5.

21. The method in accordance with claim 1 wherein the composition further comprises an emulsifier selected from the group consisting of sucrose esters, polyoxyethylene sorbitan esters, long chain alcohols, and glyceryl esters.

22. The method in accordance with claim 1 wherein the emulsifier comprises at least one glyceryl ester selected from the group consisting of glyceryl monooleate, triolein, trimyristin, tristearin, and mixtures thereof.

23. The method in accordance with claim 1 wherein the composition further comprises up to about 5 percent myrtenol, based on the total weight of the composition.

24. The method in accordance with claim 1 wherein the composition further comprises a preservative.

25. The method in accordance with claim 1 wherein the composition further comprises a topical anesthetic.

26. The method in accordance with claim 1 wherein the composition further comprises a fragrance.

27. The method in accordance with claim 1 further comprising administering the composition on demand.

28. The method in accordance with claim 1 further comprising administering the composition about 5 minutes to about 15 minutes before sexual intercourse.

29. The method in accordance with claim 1 further comprising administering the semi-solid prostaglandin E₁ composition at least twice a week.

30. The method in accordance with claim 1 further comprising administering the semi-solid prostaglandin E₁ composition every other day.

31. The method in accordance with claim 1 further comprising administering the semi-solid prostaglandin E₁ composition daily.

32. The method in accordance with claim 3 wherein the composition is packaged as a unit dose and prostaglandin E₁ is present in the amount of about 0.1 mg to about 0.5 mg per unit dose.

33. The method in accordance with claim 3 wherein the composition is packaged as a unit dose and prostaglandin E₁ is present in the amount of about 0.1 mg to about 0.3 mg per unit dose.

34. The method in accordance with claim 3 wherein the composition comprises:
about 0.001 weight percent to about 1 weight percent prostaglandin E₁;
about 0.5 weight percent to about 10 weight percent dodecyl 2-(N,N dimethylamino)-propionate hydrochloride;
about 0.5 weight percent to about 10 weight percent ethyl alcohol;
about 0.5 weight percent to about 10 weight percent ethyl laurate; and
about 0.01 weight percent to about 5 weight percent modified guar gum, based on the total weight of the composition.

35. A method of treating erectile dysfunction in a patient suffering from the co-morbid condition of diabetes mellitus comprising the step of placing in the fossa navicularis of the patient an erection-inducing amount of a semi-solid prostaglandin E₁ composition comprising:
a penetration enhancer;
a shear-thinning polymeric thickener selected from the group consisting of a polysaccharide gum and a polyacrylic acid polymer;
a component that is selected from the group consisting of an aliphatic C₁ to C₈ alcohol, an aliphatic C₈ to C₃₀ ester, and a mixture thereof; and
an acidic buffer system.

36. A method of treating erectile dysfunction in a patient suffering from the co-morbid condition of hypertension comprising the step of placing in the fossa navicularis of the patient an erection-inducing amount of a semi-solid prostaglandin E₁ composition comprising:
a penetration enhancer;
a shear-thinning polymeric thickener selected from the group consisting of a polysaccharide gum and a polyacrylic acid polymer;
a component that is selected from the group consisting of an aliphatic C₁ to C₈ alcohol, an aliphatic C₈ to C₃₀ ester, and a mixture thereof; and
an acidic buffer system.

37. A method of treating erectile dysfunction in a patient suffering from the co-morbid condition of cardiac disease comprising the step of placing in the fossa navicularis of the patient an erection-inducing amount of a semi-solid prostaglandin E₁ composition comprising:
a penetration enhancer;
a shear-thinning polymeric thickener selected from the group consisting of a polysaccharide gum and a polyacrylic acid polymer;
a component that is selected from the group consisting of an aliphatic C₁ to C₈ alcohol, an aliphatic C₈ to C₃₀ ester, and a mixture thereof; and
an acidic buffer system.

38. A method of treating erectile dysfunction in a patient having the co-morbid condition of recovering from prostatectomy comprising the step of placing in the fossa navicularis of the patient an erection-inducing amount of a semi-solid prostaglandin E₁ composition comprising:
a penetration enhancer;
a shear-thinning polymeric thickener selected from the group consisting of a polysaccharide gum and a polyacrylic acid polymer;
a component that is selected from the group consisting of an aliphatic C₁ to C₈ alcohol, an aliphatic C₈ to C₃₀ ester, and a mixture thereof; and
an acidic buffer system.

39. A method of treating a patient suffering from erectile dysfunction unresponsive to oral phosphodiesterase-5 inhibitor therapy comprising the step of placing in the fossa navicularis of the patient an erection-inducing amount of a semi-solid prostaglandin E₁ composition comprising:
a penetration enhancer;
a shear-thinning polymeric thickener selected from the group consisting of a polysaccharide gum and a polyacrylic acid polymer;
a component that is selected from the group consisting of an aliphatic C₁ to C₈ alcohol, an aliphatic C₈ to C₃₀ ester, and a mixture thereof; and
an acidic buffer system.

40. The use of prostaglandin E₁;
a penetration enhancer selected from the group consisting of an alkyl-2-(N-substituted amino)-alkanoate, an alkyl-2-(N,N-disubstituted amino)-alkanoate, an (N-substituted amino)-alkanol alkanoate, an (N,N-disubstituted amino)-alkanol alkanoate, pharmaceutically acceptable salts thereof and mixtures thereof;
a polymeric thickener selected from the group consisting of a polysaccharide gum and a polyacrylic acid polymer;
a lipophilic component selected from the group consisting of an aliphatic C₁ to C₈ alcohol, an aliphatic C₈ to C₃₀ ester, and a mixture thereof; and an acidic buffer system for the preparation of a pharmaceutical composition for treating erectile dysfunction in a patient suffering from a co-morbid condition including at least one of diabetes mellitus, hypertension, cardiac disease, history of prostatectomy or erectile dysfunction unresponsive to oral phosphodiesterase-5 inhibitor therapy, whereby the pharmaceutical composition is to be placed in the fossa navicularis of the patient.
